(19) United States
(12) Patent Application Publication
(10) Pub. No.: US 2024/0165199 A1
(43) Pub. Date: May 23, 2024

(54) PROX1 PREVENTS MYXOMATOUS VALVE DISEASE BY INHIBITING PDGF-B SIGNALING

(71) Applicant: Oklahoma Medical Research Foundation, Oklahoma City, OK (US)

(72) Inventors: YenChun Ho, Oklahoma City, OK (US); Xin Geng, Edmond, OK (US); Rajanarayanan Sathishkumar Srinivasan, Edmond, OK (US)

(21) Appl. No.: 18/506,519

(22) Filed: Nov. 10, 2023

Related U.S. Application Data
(60) Provisional application No. 63/426,581, filed on Nov. 18, 2022.

(51) Int. Cl. A61K 38/17; A61K 31/506; A61P 9/00

(52) U.S. Cl. CPC A61K 38/1709; A61K 31/506; A61P 9/00

(57) ABSTRACT

The present invention includes a method for treating a patient suffering from myxomatous mitral valve disease (MMVD), the method comprising administering to the patient an effective amount of a pharmaceutical composition comprising a Prox1 gene, a Prox1 mimic, a PDGF antagonist, or a PDGFRB antagonist that prevents the thickening of heart valves and delays the onset of clinical symptoms of myxomatous valve disease in the patient.

### CROSS-REFERENCE TO RELATED APPLICATIONS

This application is a non-provisional patent application of and claims priority to U.S. provisional patent application Ser. No. 63/426,581 filed on Nov. 18, 2022, the contents of which are incorporated by reference in its entirety.

### TECHNICAL FIELD OF THE INVENTION

The present invention relates in general to the field of treatments for myxomatous valve disease, and more particularly to the inhibition of PDGF-Beta signaling to prevent or treat myxomatous valve disease.

### STATEMENT OF FEDERALLY FUNDED RESEARCH

None.

### INCORPORATION-BY-REFERENCE OF MATERIALS FILED ON COMPACT DISC

None.

### BACKGROUND OF THE INVENTION

Without limiting the scope of the invention, its background is described in connection with myxomatous valve disease.

The present invention relates to heart valve treatments, and more particularly, to compositions and methods for treating myxomatous mitral valve disease. Properly functioning heart valves maintain unidirectional blood flow in the circulatory system by opening and closing one side of the valve to the other. The two atrioventricular valves (mitral and tricuspid valves) are multicuspid valves that prevent backflow from the ventricles into the atria during systole. The atrioventricular valves are anchored to the wall of the ventricle by chordae tendineae, which prevent the valve from inverting.

The mitral valve is located at the left ventricle and is made up of two leaflets and an incomplete diaphanous ring around the valve, known as the mitral valve annulus. When the valve opens, blood flows into the left ventricle. After the left ventricle fills with blood and contracts, the two leaflets of the mitral valve are pushed upwards and close. Closure of the mitral valve prevents blood from flowing back into the left atrium and the lungs.

Myxomatous valve disease in which the abnormal mitral valve leaflets prolapse (i.e., a portion of the affected leaflet may be billowed, loose, and floppy), which causes regurgitation. Mitral valve regurgitation is associated with increased proteoglycans.

Mitral valve prolapse causes mitral regurgitation. Isolated posterior leaflet prolapse of the human heart mitral valve, i.e., prolapse of a single leaflet, is the most common cause of mitral regurgitation. The exact cause of the prolapse are not clear, but untreated mitral regurgitation can lead to congestive heart failure and pulmonary hypertension.

As such, there is a need for treatments that prevent or treat myxomatous mitral valve disease. Among other advantages, the present invention may address one or more of these needs.

### SUMMARY OF THE INVENTION

As embodied and broadly described herein, an aspect of the present disclosure relates to a method for treating a patient suffering from myxomatous mitral valve disease (MMVD), the method comprising administering to the patient an effective amount of a pharmaceutical composition comprising: a Prox1 gene, a Prox1 mimic, a Platelet Derived Growth Factor (PDGF) antagonist or a Platelet Derived Growth Factor Receptor Beta (PDGFRB) antagonist that prevents the thickening of heart valves and delays the onset of clinical symptoms of myxomatous valve disease in the patient. In one aspect, the PDGF or PDGFRB antagonist is selected from at least one of: AC710, AC710 Mesylate, AG1295, AG1296, an antagonistic human monoclonal or portion thereof targeting PDGFRB, an antagonistic human monoclonal or portion thereof targeting PDGF, avapritinib, axitinib, AZD2932, BOT-191, cediranib, celecoxib, CP 673451, crenolanib, dasatinib, Desethyl Sunitinib, DMPQ dihydrochloride, dovitinib, etoricoxib and DFU, ilorasertib, imatinib, imatinib mesylate, KG 5, lenvatinib, Linifanib, N-CP-673451, nilotinib, nintedanib, orantinib, pazopanib, PDGFRa kinase inhibitor-1, ponatibib, radotinib, regorafenib, ripretinib, sorafenib, SU 4312, SU 5402, SU14813, SU14813 maleate, SU16f, sunitinib, sunitinib malate, TAK 593, TAK-593, TG 100572, toceranib, or toceranib phosphate. In another aspect, the Prox1 gene or Prox1 mimic is an RNA, a DNA, or derivatives thereof. In another aspect, the PDGFRB antagonist is imatinib. In another aspect, the method further comprises adding one or more non-active pharmaceutically acceptable ingredients selected from at least one of: buffers, excipients, binders, diluents, vehicles, lubricants, wetting, emulsifying, salts, or carriers. In another aspect, the Prox1 gene, Prox1 mimic, PDGF antagonist, or PDGFRB antagonist is administered orally in the form of a tablet or a capsule. In another aspect, the administration of the Prox1 gene, Prox1 mimic, PDGF antagonist, or PDGFRB antagonist results in one or more of the following in the patient selected from the group consisting of: effects a prolongation of the preclinical phase without exhibiting clinical symptoms of heart failure, effects a delay of onset of clinical symptoms of heart failure, increases the survival time of the treated patient as compared to placebo treatment, improves the quality of life of the treated patient, improves cardiac function/output in the treated patient, reduces sudden cardiac death of the patient due to cardiac reasons, and reduces the risk of reaching heart failure. In another aspect, the patient is a mammal selected from the group consisting of: a human, a dog, a cat, and a horse. In another aspect, the Prox1 gene, Prox1 mimic, PDGF antagonist, or PDGFRB antagonist is administered in a daily dose of 0.1, 0.5, 1, 2, 3, 4, 5, 6, 7, 8, 9 10, 12.5, 15, 20, 25, 30, 40 50, 60, 75, 80, 90 or 100 mg/kg bodyweight. In another aspect, the daily dose of the Prox1 gene, Prox1 mimic, PDGF antagonist, or PDGFRB antagonist is administered as two doses 0.05, 0.5, 1, 2, 3, 4, 5, 6, 7, 8, 9 10, 12.5, 15, 20, 25, 30, 40, or 50 mg/kg bodyweight administered every 12 hours. In another aspect, the daily dose of the PDGF inhibitor imatinib is between 100, 200, 250, 300, 400, 500, 600, 700, 750, or 800 mg per day. In another aspect, the Prox1 gene, Prox1 mimic, PDGF antagonist, or PDGFRB antagonist is administered orally, intravenously, enterally, or parenterally. In another aspect, the administration of the Prox1 gene, Prox1 mimic, PDGF antagonist, or PDGFRB antagonist further effects a prolongation of the time of survival of the patient, as compared to placebo treatment or non-PDGFRB antagonist treatment, of at least about 30 days, at least about 5 months, or at least about 7 months.

As embodied and broadly described herein, an aspect of the present disclosure relates to a method for detecting and treating a patient suffering from myxomatous mitral valve disease (MMVD), the method comprising: identifying that the patient has at least one of: mutation or deletion of Prox1, platelet derived growth factor (PDGF) secretion is unregulated, or PDGF receptor beta (PDGFRB) signaling is unregulated; and administering to the patient an effective amount of a pharmaceutical composition comprising at least one of: a Prox1 gene, a Prox1 mimic, a PDGF antagonist, or a PDGFRB antagonist that prevents the thickening of heart valves and delays the onset of clinical symptoms of myxomatous valve disease in the patient. In one aspect, the PDGF or PDGFRB antagonist is selected from at least one of: AC710, AC710 Mesylate, AG1295, AG1296, an antagonistic human monoclonal or portion thereof targeting PDGFRB, an antagonistic human monoclonal or portion thereof targeting PDGF, avapritinib, axitinib, AZD2932, BOT-191, cediranib, celecoxib, CP 673451, crenolanib, dasatinib, Desethyl Sunitinib, DMPQ dihydrochloride, dovitinib, etoricoxib and DFU, ilorasertib, imatinib, imatinib mesylate, KG 5, lenvatinib, Linifanib, N-CP-673451, nilotinib, nintedanib, orantinib, pazopanib, PDGFRa kinase inhibitor-1, ponatibib, radotinib, regorafenib, ripretinib, sorafenib, SU 4312, SU 5402, SU14813, SU14813 maleate, SU16f, sunitinib, sunitinib malate, TAK 593, TAK-593, TG 100572, toceranib, or toceranib phosphate. In another aspect, the Prox1 gene or Prox1 mimic is an RNA, a DNA, or a derivative thereof. In another aspect, the PDGFRB antagonist is imatinib. In another aspect, the method further comprises adding one or more non-active pharmaceutically acceptable ingredients selected from at least one of: buffers, excipients, binders, diluents, vehicles, lubricants, wetting, emulsifying, salts, or carriers. In another aspect, the Prox1 gene, Prox1 mimic, PDGF antagonist, or PDGFRB antagonist is administered orally in the form of a tablet or a capsule. In another aspect, the administration of the Prox1 gene, Prox1 mimic, PDGF antagonist, or PDGFRB antagonist results in one or more of the following in the patient selected from the group consisting of: effects a prolongation of the preclinical phase without exhibiting clinical symptoms of heart failure, effects a delay of onset of clinical symptoms of heart failure, increases the survival time of the treated patient as compared to placebo treatment, improves the quality of life of the treated patient, improves cardiac function/output in the treated patient, reduces sudden cardiac death of the patient due to cardiac reasons, and reduces the risk of reaching heart failure. In another aspect, the patient is a mammal selected from the group consisting of: a human, a dog, a cat, and a horse. In another aspect, the Prox1 gene, Prox1 mimic, PDGF antagonist, or PDGFRB antagonist is administered in a daily dose of 0.1, 0.5, 1, 2, 3, 4, 5, 6, 7, 8, 9 10, 12.5, 15, 20, 25, 30, 40 50, 60, 75, 80, 90 or 100 mg/kg bodyweight. In another aspect, the daily dose of the Prox1 gene, Prox1 mimic, PDGF antagonist, or PDGFRB antagonist is administered as two doses 0.05, 0.5, 1, 2, 3, 4, 5, 6, 7, 8, 9 10, 12.5, 15, 20, 25, 30, 40, or 50 mg/kg bodyweight administered every 12 hours. In another aspect, the daily dose of the PDGF inhibitor imatinib is between 100, 200, 250, 300, 400, 500, 600, 700, 750, or 800 mg per day. In another aspect, the Prox1 gene, Prox1 mimic, PDGF antagonist, or PDGFRB antagonist is administered orally, intravenously, enterally, or parenterally. In another aspect, the administration of the Prox1 gene, Prox1 mimic, PDGF antagonist, or PDGFRB antagonist further effects a prolongation of the time of survival of the patient, as compared to placebo treatment or non-PDGFRB antagonist treatment, of at least about 30 days, at least about 5 months, or at least about 7 months.

As embodied and broadly described herein, an aspect of the present disclosure relates to a method for preventing suffering from myxomatous mitral valve disease (MMVD) in a human patient, the method comprising administering to the human patient an effective amount of a pharmaceutical composition comprising a Platelet Derived Growth Factor (PDGF) antagonist or a Platelet Derived Growth Factor Receptor Beta (PDGFRB) antagonist that prevents the thickening of heart valves and delays the onset of clinical symptoms of myxomatous valve disease in the patient.

### BRIEF DESCRIPTION OF THE DRAWINGS

For a more complete understanding of the features and advantages of the present invention, reference is now made to the detailed description of the invention along with the accompanying figures and in which:

FIGS. 1A to 1C shows the deletion of Prox1 (prospero-related homeobox transcription factor 1) from valvular endothelial cells (VECs) results in progressive myxomatous valve degeneration and aortic valve (AV) stenosis.

FIGS. 2A to 2D show that the loss of PROX1 (Prospero-related homeobox transcription factor 1) from aortic valvular endothelial cells (VECs) results in valve thickening, damaged endothelium, thrombus formation, and downregulation of FOXC2 (Forkhead box C2 transcription factor) in VECs.

FIGS. 3A to 3G show that increased proteoglycan expression and disrupted collagen and elastin fibers are observed in the aortic valves of Prox1^{ΔVEC} mice.

FIGS. 4A to 4D show that hyperactivation of PDGF-B (platelet-derived growth factor-B)/PDGFRβ (PDGF receptor 3) signaling causes myxomatous degeneration of aortic valve.

FIGS. 5A to 5H shows that knock down of FOXC2 (Forkhead box C2 transcription factor) results in enlarged aortic valves, and over expression of FOXC2 rescues the aortic valve defects of Prox1^{ΔVEC} mice.

FIG. 6A to 6F show that PDGF-B (platelet-derived growth factor-B)/PDGFRβ (PDGF receptor β) signaling promotes proteoglycan expression in valvular interstitial cells (VICs) in a SOX9 (SRY-related HMG-box 9)-dependent manner.

FIGS. 7A to 7F show that PDGF-B (platelet-derived growth factor-B) and SOX9 (SRY-related HMG-box 9) are increased in the myxomatous valves from human patients.

FIG. 8A to 8E show that the receptor tyrosine kinase (RTK) inhibitor imatinib (Imb) partially rescues aortic valve function in Prox1^{ΔVEC} mice.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Abbreviations: AV, aortic valve; MV, mitral valve; A, aorta; V, left ventricle; AVI, leaflet with aortic valve insufficiency; Veh, vehicle; Imb, imatinib; PDGF, platelet derived growth factor; PDGFR-B or PDGFR-beta, platelet derived growth factor receptor beta.

The present inventors discovered that Prospero Homeobox Protein 1 (Prox1) (NCBI Entrez Gene: 5629, UniProtKB/Swiss-Prot: Q92786, HGNC: 9459, relevant sequence incorporated herein by reference) is expressed in a subset of cardiac valve endothelial cells. It was found that the deletion of Prox1 from valve cells results in myxomatous cardiac valves in which platelet derived growth factor (PDGF) secretion is unregulated and/or PDGF receptor beta (PDGFR-B)(HGNC: 8804, NCBI Entrez Gene: 5159, UniProtKB/Swiss-Prot: P09619) signaling is unregulated. It was further determined that the inhibition of PDGF-B signaling with imatinib prevents the progression of valve disease in mice lacking Prox1. Finally, it was found that PDGFR-B inhibition prevents the progression of valve disease caused by Prox1 deficiency. Thus, the present invention includes determining if there is a deficiency in Prox1 and/or an increase in PDGFR-B signaling and treating with the PDGF antagonist to prevent or treat myxomatous valve disease and/or mitral valve prolapse.

As used herein, the terms “inhibitor,” “antagonist” or “downregulator” are used interchangeably and refer to a substance that results in a detectably lower expression or activity level as compared to a control. The inhibited expression or activity can be 10%, 20%, 30%, 40%, 50%, 60%, 70%, 80%, 90% or less than that in a control. In certain instances, the inhibition is 1.5-fold, 2-fold, 3-fold, 4-fold, 5-fold, 10-fold, or more in comparison to a control. An “inhibitor” is a peptide, siRNA, (e.g., shRNA, miRNA, snoRNA), compound or small molecule that inhibits cellular function (e.g., replication) e.g., by binding, partially or totally blocking stimulation, decrease, prevent, or delay activation, or inactivate, desensitize, or down-regulate signal transduction, gene expression or enzymatic activity necessary for protein activity.

As used herein, the terms “an effective amount” or “a therapeutically effective amount” refers to an amount sufficient to accomplish a stated purpose (e.g. achieve the effect for which it is administered, prevent a disease, treat a disease, reduce protein tyrosine kinase activity, reduce transcriptional activity (e.g., PDGF), increase transcriptional activity (e.g., Prox1), reduce one or more symptoms of a disease or condition (myxomatous valve disease and/or mitral valve prolapse). An example of an “effective amount” is an amount sufficient to contribute to the treatment, prevention, or reduction of a symptom or symptoms of a disease, which could also be referred to as a “therapeutically effective amount.” The exact amounts will depend on the effectiveness of the treatment and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins), relevant portions incorporated herein by reference.

In one aspect, the effective amount of the Prox1 gene, Prox1 mimic, PDGF antagonist, or PDGFRB antagonist can be administered in a daily dose of 0.1, 0.5, 1, 2, 3, 4, 5, 6, 7, 8, 9 10, 12.5, 15, 20, 25, 30, 40 50, 60, 75, 80, 90 or 100 mg/kg bodyweight. In another aspect, the daily dose of the Prox1 gene, Prox1 mimic, PDGF antagonist, or PDGFRB antagonist is administered as two or more doses 0.05, 0.5, 1, 2, 3, 4, 5, 6, 7, 8, 9 10, 12.5, 15, 20, 25, 30, 40, or 50 mg/kg bodyweight administered every 12 hours. In another aspect, the daily dose of the PDGF inhibitor imatinib is between 100, 200, 250, 300, 400, 500, 600, 700, 750, or 800 mg per day.

As used herein, the term “reduction” of a symptom or symptoms (and grammatical equivalents of this phrase) refers to decreasing of the severity or frequency of the symptom(s), or elimination of the symptom(s).

As used herein, the term “a prophylactically effective amount” of a drug refers to an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms.

For example, for use with the present invention a PDGF or a PDGFRB antagonist can be selected from at least one of: AC710, AC710 Mesylate, AG1295, AG1296, an antagonistic human monoclonal or portion thereof targeting PDGFRB, an antagonistic human monoclonal or portion thereof targeting PDGF, avapritinib, axitinib, AZD2932, BOT-191, cediranib, celecoxib, CP 673451, crenolanib, dasatinib, Desethyl Sunitinib, DMPQ dihydrochloride, dovitinib, etoricoxib and DFU, ilorasertib, imatinib, imatinib mesylate, KG 5, lenvatinib, Linifanib, N-CP-673451, nilotinib, nintedanib, orantinib, pazopanib, PDGFRa kinase inhibitor-1, ponatibib, radotinib, regorafenib, ripretinib, sorafenib, SU 4312, SU 5402, SU14813, SU14813 maleate, SU16f, sunitinib, sunitinib malate, TAK 593, TAK-593, TG 100572, toceranib, or toceranib phosphate.

A dosage unit for use of the PDGF and/or PDGFRB antagonists of the present invention, may be a single compound or mixtures thereof with other compounds. The compound may be mixed together, form ionic or even covalent bonds. The PDGF and/or PDGFRB antagonists of the present invention may be administered in oral, intravenous (bolus or infusion), intraperitoneal, subcutaneous, or intramuscular form, all using dosage forms well known to those of ordinary skill in the pharmaceutical arts. Depending on the particular location or method of delivery, different dosage forms, e.g., tablets, capsules, pills, powders, granules, elixirs, tinctures, suspensions, syrups, and emulsions may be used to provide the PDGF and/or PDGFRB antagonists of the present invention to a patient in need of therapy that includes the PDGF and/or PDGFRB antagonists. The PDGF and/or PDGFRB antagonists may also be administered as any one of known salt forms.

PDGF and/or PDGFRB antagonists are typically administered in admixture with suitable pharmaceutical salts, buffers, diluents, extenders, excipients and/or carriers (collectively referred to herein as a pharmaceutically acceptable carrier or carrier materials) selected based on the intended form of administration and as consistent with conventional pharmaceutical practices. Depending on the best location for administration, the PDGF and/or PDGFRB antagonists may be formulated to provide, e.g., maximum and/or consistent dosing for the particular form for oral, rectal, topical, intravenous injection or parenteral administration. While the PDGF and/or PDGFRB antagonists may be administered alone, it will generally be provided in a stable salt form mixed with a pharmaceutically acceptable carrier. The carrier may be solid or liquid, depending on the type and/or location of administration selected.

Techniques and compositions for making useful dosage forms using the present invention are described in one or more of the following references: Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2007; Goodman and Gilman, eds., The Pharmacological Basis of Therapeutics, Tenth Edition, McGraw Hill, 2001; Remington's Pharmaceutical Sciences, 20th Ed., Lippincott Williams & Wilkins., 2000, and updates thereto; Martindale, The Extra Pharmacopoeia, Thirty-Second Edition (The Pharmaceutical Press, London, 1999); all of which are incorporated by reference, and the like, relevant portions incorporated herein by reference.

For example, the PDGF and/or PDGFRB antagonists may be included in a tablet. Tablets may contain, e.g., suitable binders, lubricants, disintegrating agents, coloring agents, flavoring agents, flow-inducing agents and/or melting agents. For example, oral administration may be in a dosage unit form of a tablet, gelcap, caplet or capsule, the active drug component being combined with a non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol, mixtures thereof, and the like. Suitable binders for use with the present invention include: starch, gelatin, natural sugars (e.g., glucose or beta-lactose), corn sweeteners, natural and synthetic gums (e.g., acacia, tragacanth or sodium alginate), carboxymethylcellulose, polyethylene glycol, waxes, and the like. Lubricants for use with the invention may include: sodium oleate, sodium stearate, magnesium stearate, sodium benzoate, sodium acetate, sodium chloride, mixtures thereof, and the like. Disintegrators may include: starch, methyl cellulose, agar, bentonite, xanthan gum, mixtures thereof, and the like.

The PDGF and/or PDGFRB antagonists may be administered in the form of liposome delivery systems, e.g., small unilamellar vesicles, large unilamallar vesicles, and multilamellar vesicles, whether charged or uncharged. Liposomes may include one or more: phospholipids (e.g., cholesterol), stearylamine and/or phosphatidylcholines, mixtures thereof, and the like.

The PDGF and/or PDGFRB antagonists may also be coupled to one or more soluble, biodegradable, bioacceptable polymers as drug carriers or as a prodrug. Such polymers may include: polyvinylpyrrolidone, pyran copolymer, polyhydroxylpropylmethacrylamide-phenol, polyhydroxyethylasparta-midephenol, or polyethyleneoxide-polylysine substituted with palmitoyl residues, mixtures thereof, and the like. Furthermore, the PDGF and/or PDGFRB antagonists may be coupled one or more biodegradable polymers to achieve controlled release of the PDGF and/or PDGFRB antagonists, biodegradable polymers for use with the present invention include: polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels, mixtures thereof, and the like.

In one embodiment, gelatin capsules (gelcaps) may include the PDGF and/or PDGFRB antagonists and powdered carriers, such as lactose, starch, cellulose derivatives, magnesium stearate, stearic acid, and the like. Like diluents may be used to make compressed tablets. Both tablets and capsules may be manufactured as immediate-release, mixed-release or sustained-release formulations to provide for a range of release of medication over a period of minutes to hours. Compressed tablets may be sugar-coated or film-coated to mask any unpleasant taste and protect the tablet from the atmosphere. An enteric coating may be used to provide selective disintegration in, e.g., the gastrointestinal tract.

For oral administration in a liquid dosage form, the oral drug components may be combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents, mixtures thereof, and the like.

Liquid dosage forms for oral administration may also include coloring and flavoring agents that increase patient acceptance and therefore compliance with a dosing regimen. In general, water, a suitable oil, saline, aqueous dextrose (e.g., glucose, lactose and related sugar solutions) and glycols (e.g., propylene glycol or polyethylene glycols) may be used as suitable carriers for parenteral solutions. Solutions for parenteral administration include generally, a water soluble salt of the active ingredient, suitable stabilizing agents, and if necessary, buffering salts. Antioxidizing agents such as sodium bisulfite, sodium sulfite and/or ascorbic acid, either alone or in combination, are suitable stabilizing agents. Citric acid and its salts and sodium EDTA may also be included to increase stability. In addition, parenteral solutions may include pharmaceutically acceptable preservatives, e.g., benzalkonium chloride, methyl or propyl-paraben, and/or chlorobutanol. Suitable pharmaceutical carriers are described in Remington's Pharmaceutical Sciences, Mack Publishing Company, a standard reference text in this field, relevant portions incorporated herein by reference.

For direct delivery to the nasal passages, sinuses, mouth, throat, esophagus, trachea, lungs, and alveoli, the PDGF and/or PDGFRB antagonists may also be delivered as an intranasal form by use of a suitable intranasal vehicle. For dermal and transdermal delivery, the PDGF and/or PDGFRB antagonists may be delivered using lotions, creams, oils, elixirs, serums, transdermal skin patches, and the like, as are well known to those of ordinary skill in that art. Parenteral and intravenous forms may also include pharmaceutically acceptable salts and/or minerals and other materials to make them compatible with the type of injection or delivery system chosen, e.g., a buffered, isotonic solution. Examples of useful pharmaceutical dosage forms for the administration of PDGF and/or PDGFRB antagonists may include the following forms.

Capsules. Capsules may be prepared by filling standard two-piece hard gelatin capsules each with 10 to 500 milligrams of powdered active ingredient, 5 to 150 milligrams of lactose, 5 to 50 milligrams of cellulose, and 6 milligrams magnesium stearate.

Soft Gelatin Capsules. A mixture of active ingredients is dissolved in a digestible oil such as soybean oil, cottonseed oil, or olive oil. The active ingredient is prepared and injected by using a positive displacement pump into gelatin to form soft gelatin capsules containing, e.g., 100-500 milligrams of the active ingredient. The capsules are washed and dried.

Tablets. A large number of tablets are prepared by conventional procedures so that the dosage unit was 100-500 milligrams of the active ingredient, 0.2 milligrams of colloidal silicon dioxide, 5 milligrams of magnesium stearate, 50-275 milligrams of microcrystalline cellulose, 11 milligrams of starch and 98.8 milligrams of lactose. Appropriate coatings may be applied to increase palatability or delay absorption.

To provide an effervescent tablet appropriate amounts of, e.g., monosodium citrate and sodium bicarbonate, are blended together and then roller compacted, in the absence of water, to form flakes that are then crushed to give granulates. The granulates are then combined with the active ingredient, drug and/or salt thereof, conventional beading or filling agents and, optionally, sweeteners, flavors and lubricants.

Injectable solution. A parenteral composition suitable for administration by injection is prepared by stirring 1.5% by weight of active ingredient in deionized water and mixed with, e.g., up to 10% by volume propylene glycol and water. The solution is made isotonic with sodium chloride and sterilized using, e.g., ultrafiltration.

Suspension. An aqueous suspension is prepared for oral administration so that each 5 ml contains 100 mg of finely divided active ingredient, 200 mg of sodium carboxymethyl cellulose, 5 mg of sodium benzoate, 1.0 g of sorbitol solution, U.S.P., and 0.025 ml of vanillin.

For mini tablets, the active ingredient is compressed into a hardness in the range 6 to 12 Kp. The hardness of the final tablets is influenced by the linear roller compaction strength used in preparing the granulates, which are influenced by the particle size of, e.g., the monosodium hydrogen carbonate and sodium hydrogen carbonate. For smaller particle sizes, a linear roller compaction strength of about 15 to 20 KN/cm may be used.

Kits. The present invention also includes pharmaceutical kits useful, for example, for the treatment of cancer, which comprise one or more containers containing a pharmaceutical composition comprising a therapeutically effective amount of PDGF and/or PDGFRB antagonists. Such kits may further include, if desired, one or more of various conventional pharmaceutical kit components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components, may also be included in the kit. It should be understood that although the specified materials and conditions are important in practicing the invention, unspecified materials and conditions are not excluded so long as they do not prevent the benefits of the invention from being realized.

Examples of suitable liquid dosage forms include solutions or suspensions in water, pharmaceutically acceptable fats and oils, alcohols, or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

A chewable form refers to semi-soft, palatable, and stable chewable treat without the addition of water. It should be appreciated to the skilled artisan that a chewable composition will be stable and palatable, fast disintegrating, semi-soft medicated chewable tablets (treats) by extrusion without the addition of extraneous water. A soft chewable tablet does not harden on storage and is resistant to microbial contamination. A semi-soft chewable contains a blend of any one or more binders, flavors, palatability enhancers, humectants, disintegrating agents, non-aqueous solvents, and diluents that are plasticized with liquid plasticizers, such as glycols and polyols to make them ductile and extrudable. The chewable can be made by extrusion, e.g., including fats or lipids as plasticizers and binding agents, is manufactured in the absence of added water, uses plasticizers to replace water in extrudable matrices, contains humectants to maintain the extrudable chew in a pliant and soft state during its shelf life, or any combination thereof. The chewable form may be provided in conjunction with one or more flavorants and/or taste masking agents that improve the taste of the formulation greater than 10, 20, 30, 40, 50, 60, 70, 80, or 90%. The chewable can include the active agent and the ion exchange resin to enhance taste masking.

The present invention is demonstrated by the following results.

The inventors have previously shown that PROX1 is expressed in a subset of CD31⁺ (cluster of differentiation 31⁺) VECs that were located on the downstream side (fibrosa) of aortic valves at embryonic day (E) 16.5.8 The inventors analyzed embryos at earlier time points and found that PROX1 was not expressed in the atrioventricular cushion of E10.5 embryos. However, PROX1 was expressed in the VECs of heart valves at least as early as E12.5. At E12.5 PROX1 expression did not appear to be obviously polarized. However, PROX1 expression became progressively restricted to the downstream VECs at subsequent time points and remained so in adult valves. The inventors generated a new Prox1-2A-Cre mouse model that expresses Cre recombinase from the regulatory elements of Prox1 without compromising PROX1 expression. The inventors performed lineage tracing using this Cre line and determined that in postnatal day 7 Prox1-2A-Cre; mT/mG pups both upstream and downstream VECs were GFP⁺. This confirmed that PROX1 is initially expressed in both the upstream and downstream VECs. Additionally, it was observed that only a few GFP⁺ VICs in the Prox1-2A-Cre; mT/mG pups. VICs originate from VECs through endothelial to mesenchymal transition (EndMT) before E10.5.¹² Hence, the lineage tracing result indicates that PROX1 expression in the heart valves starts at the conclusion of EndMT.

Prox1^{-/-} embryos died at E14.5 due to the absence of lymphatic vasculature. At E14 the aortic valves of Prox1^{-/-} embryos were indistinguishable from those in their wild-type littermates. Thus, PROX1 is not necessary for EndMT and the subsequent formation of heart valve leaflets.

To study the potential role of PROX1 in heart valve development, Prox1 was specifically deleted from VECs by using the Prox1^{f/f} mice without affecting the lymphatic.¹³ To this end, the inventors used Nfatc1 (Nuclear factor of activated T cells 1)-enhancer Cre (Nfatc1^{emCre}) that is expressed specifically in the downstream VECs of heart valves.¹⁴ Analysis of the aortic valves of postnatal day 7 Nfatc1^{emCre}; Prox1^{f/f} pups revealed that the deletion of Prox1 was incomplete as indicated by the residual PROX1 expression. Nevertheless, the aortic valves of 6-month-old Nfatc1^{emCre}; Prox1^{f/f} mice showed a trend towards thickening.

The inventors searched for alternative Cre lines to efficiently delete Prox1 from VECs and investigate the valve phenotype thoroughly. In the Notch1-Cre^{Lo} mice, Cre recombinase replaces the Notch1 receptor intracellular domain at 1 Notch1 locus.¹⁵ Cre is nuclear and active in tissues with high Notch signaling, such as in arteries and heart valves, but not in veins.¹⁵ The inventors hypothesized that Notch1-Cre^{Lo} will not be active in the lymphatic vasculature, as it originates predominantly from the embryonic veins. As anticipated, lineage tracing revealed that Notch1-Cre^{Lo} was active in VECs and a substantial number of VICs, but not in the lymphatic vessels of the mesentery or skin, and venous valves.

Although heterozygous loss-of-function mutations in NOTCH1 are associated with bicuspid aortic valves and calcific aortic valve disease in humans,¹⁶ several reports have shown that the heart valves of Notch1^{-/-} mice are phenotypically normal.^{5,17-19} Consistent with these reports, 6-month-old Notch1-Cre^{Lo} mice, which are heterozygous for Notch1, had phenotypically normal cardiac valves as evaluated by histological quantification of valve thickness. Notch1-Cre^{Lo} mice also showed normal aortic valve function at 12 months of age.

Notch1-Cre^{Lo}; Prox1^{f/f} mice (referred to as Prox1^{ΔVEC} for Prox1 deletion in VECs) were born at the expected Mendelian ratio and did not display obvious lymphatic vascular defects such as edema, chylous ascites, or chylothorax. GFP is expressed from the Prox1 locus once the floxed allele is deleted by Cre recombinase.¹³ Compared with controls, Prox1^{ΔVEC} mice expressed GFP in VECs and had significantly reduced numbers of PROX1-positive VECs in the aortic and mitral valves, consistent with loss of Prox1 in VECs. Notably, Prox1^{ΔVEC} mice did not display bicuspid aortic valves. Furthermore, analysis of the heart valves of Prox1^{ΔVEC} mice by quantitative real-time polymerase chain reaction and Western blotting did not reveal any obvious changes in the expression of receptors, ligands, or targets of Notch signaling. Thus, Notch signaling is not affected in an obvious manner in the Prox1^{ΔVEC} mice.

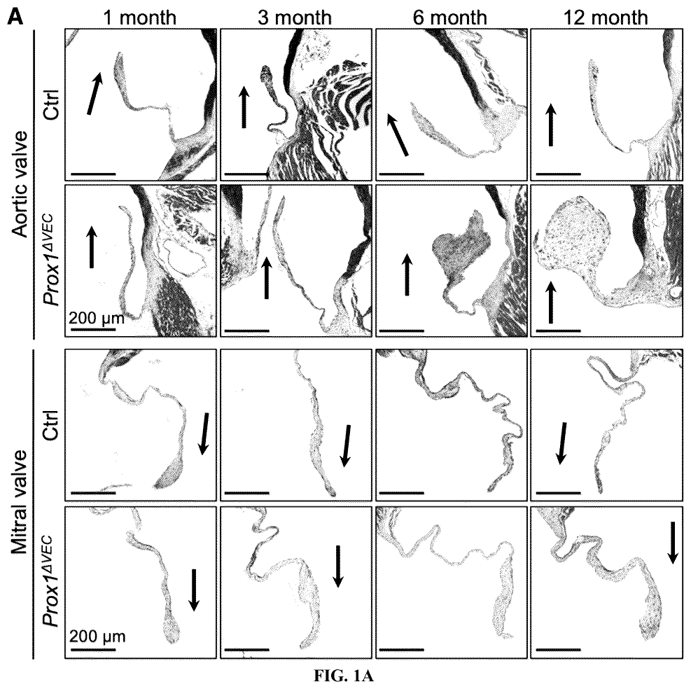

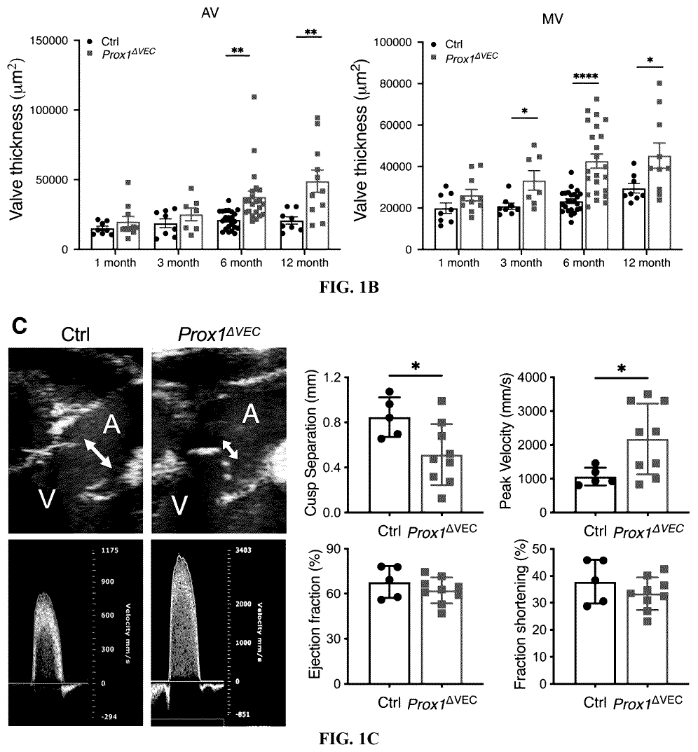

To examine heart valve thickness in adult mice, sections were stained with Movat pentachrome (FIG. 1A). Prox1^{ΔVEC} animals started to display thickened aortic and mitral valves when they were 3-month-old and their valves were significantly thicker than those from wild-type, Notch1-Cre^{Lo} or Notch1-Cre^{Lo}; Prox1^{+/f} littermates (henceforth combined as controls), at 6 and 12 months of age (FIG. 1A and FIG. 1i). The inventors reanalyzed the data from 6-month-old mice and determined that both male and female Prox1^{ΔVEC} mice develop enlarged valves. Ultrasound image analysis revealed that the cusp separation and fractional valve opening of the aortic valves were reduced in 12-month-old Prox1^{ΔVEC} mice when compared with the controls. Furthermore, color Doppler imaging revealed an increase in outflow velocity (FIG. 1C). These echocardiography studies indicated that the Prox1^{ΔVEC} mice had aortic valve stenosis phenotype. Taken together, loss of PROX1 in VECs resulted in progressive thickening of aortic and mitral valves and aortic valve stenosis in a Notch signaling-independent and sex-independent manner.

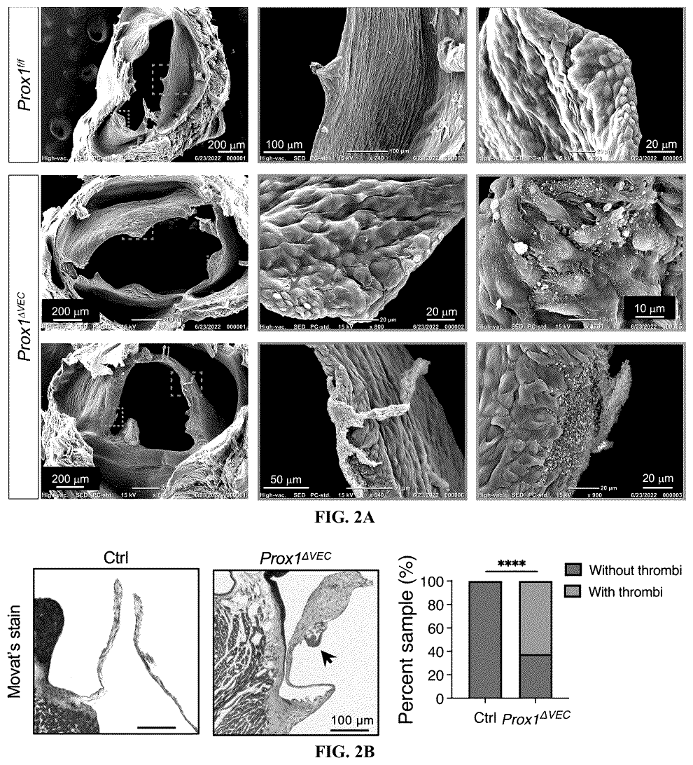

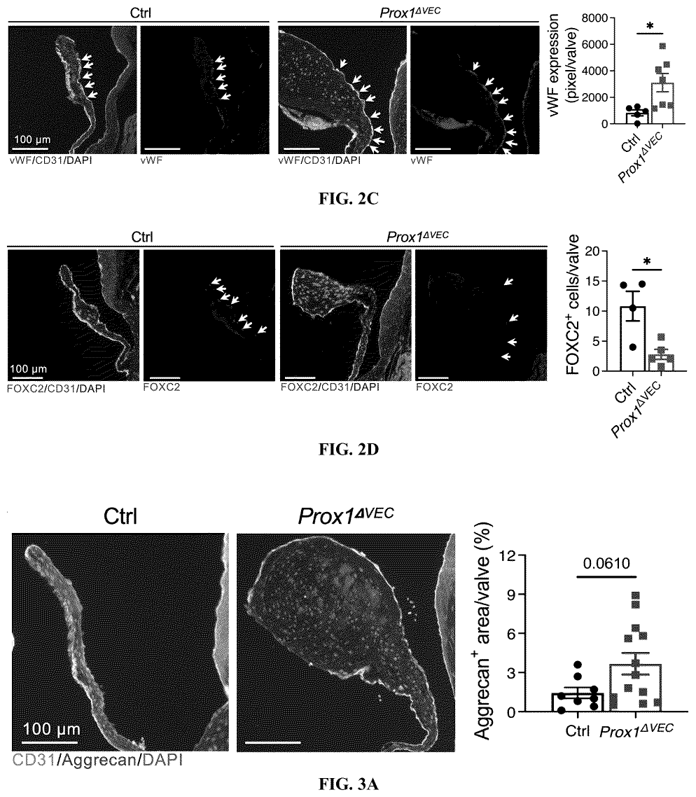

The inventors performed scanning electron microscopy to visualize the outflow side of the aortic and mitral valves. Scanning electron microscopy confirmed that the Prox1^{ΔVEC} mice do not develop bicuspid aortic valves (FIG. 2A). In both control and mutant mice, VECs along the edges of the valves were elongated and those at the sites of coaptation had a cobblestone morphology (FIG. 2A). Prox1^{ΔVEC} mice frequently showed disrupted cell junctions, detachment of the VEC layer, and platelet aggregate-like structures at the sites of coaptation in aortic valves (FIG. 2A) but not in the mitral valves. In Movat pentachrome-stained thin sections the inventors observed platelet aggregate like structures on the upstream (inflow) side of mutant aortic valves and in some mutant mitral valve (FIG. 2B). Additionally, the expression of VWF (von Willebrand Factor), a potent regulator o coagulation, was significantly increased in the aortic and mitral valves of Prox1^{ΔVEC} mice (FIG. 2C) Both PROX1 and FOXC2 are known to inhibit thrombosis in venous valves.²⁰ The inventors analyze the valves of Prox1^{ΔVEC} mice and determined that the expression of FOXC2 was significantly downregulated (FIG. 2D). Thus, PROX1 regulates the expression of FOXC2 in VECs. Platelet aggregate-like structures are observed on both the downstream (PROX1⁺) and upstream (PROX1⁻) sides of cardiac valves, and these defects are more frequently observed in aortic valves. High shear stress on the tips and upstream side of abnormally thick Prox1^{ΔVEC} aortic valves compromises VEC integrity.

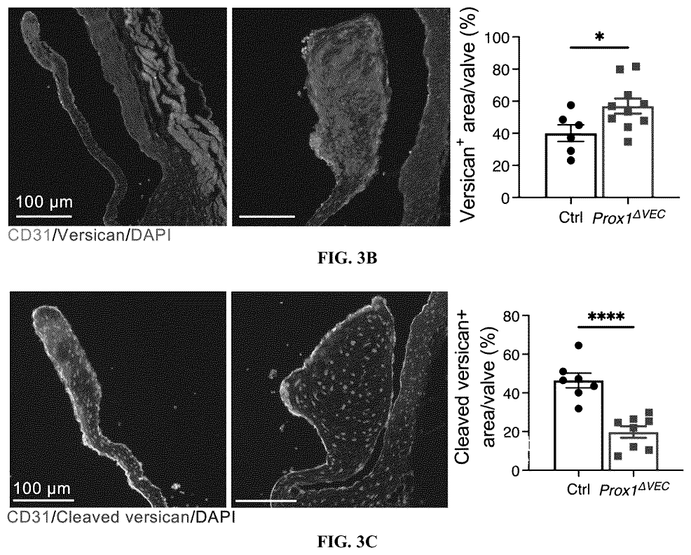

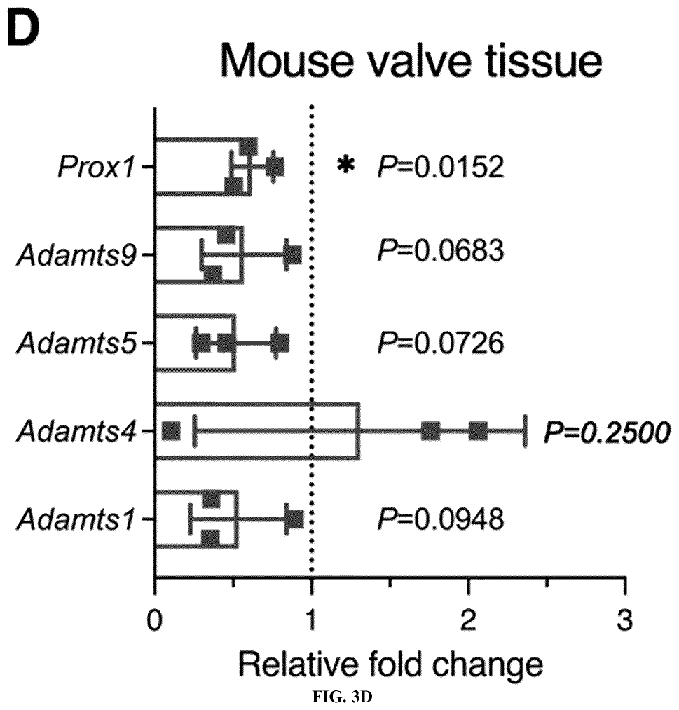

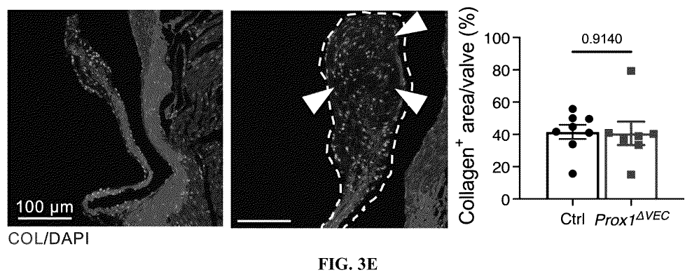

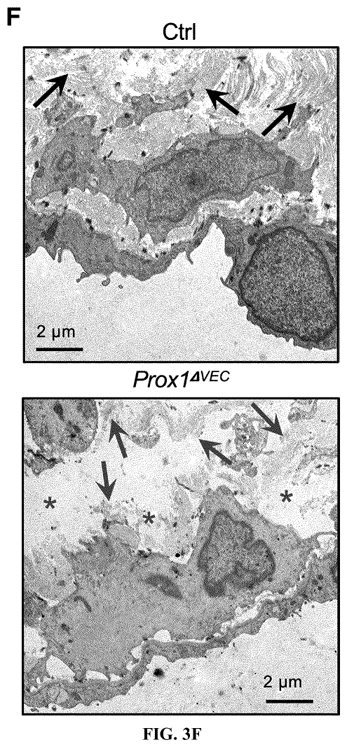

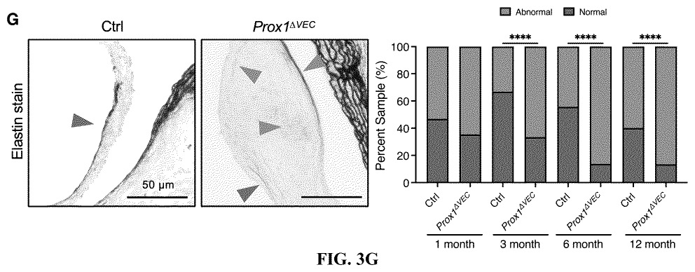

Abnormal ECM composition is a defining characteristic of valve diseases.² Hence, the inventors investigated if the expression of collagen, elastin, and proteoglycans were altered in the thickened valves of Prox1^{ΔVEC} mice. Movat pentachrome staining revealed that proteoglycan expression (staining adjacent the arrows) is increased in the valves of Prox1^{ΔVEC} mice compared with controls (FIG. 1A). Consistent with this observation, the inventors observed elevated expression of the proteoglycans aggrecan and versican in the aortic (FIG. 3A and FIG. 3B) and mitral valves of Prox1^{ΔVEC} mice.

FIGS. 1A to 1C shows the deletion of Prox1 (prospero-related homeobox transcription factor 1) from valvular endothelial cells (VECs) results in progressive myxomatous valve degeneration and aortic valve (AV) stenosis. FIG. 1A, Representative Movat Pentachrome-stained histological images of the AVs and mitral valves (MVs) of control and Prox1ΔVEC mice at various ages. Glycosaminoglycans, collagen, and elastin are stained in light gray, dark grey, and black, respectively. FIG. 1B, Quantification of AV and MV thickness. For each of the mice, 4 to 7 sections at 100-120 μm intervals were analyzed. The area of the valve leaflets (2 leaflets from base to tip) was measured for every section and their average was used as a measure of valve thickness. Each dot represents an individual mouse, 1-month old mice: n=8 controls and n=10 Prox1^{ΔVEC}; 3-month-old mice: n=8 controls and n=7 Prox1^{ΔVEC}; 6-month-old mice: n=23 controls and Prox1^{ΔVEC}; 12-month-old mice: n=8 controls and n=11 Prox1^{ΔVEC}. Data is represented as mean±SEM. FIG. 1C, Echocardiography of 12-month-old control and Prox1^{ΔVEC} mice. Left, representative echocardiography images show decreased cusp separation (double-headed arrows in the top) and increased aortic peak velocity (bottom) in Prox1ΔVEC mice. Right, Bar charts of cusp separation and AV peak velocity. No obvious defects were observed in left ventricle (LV) functions in the Prox1^{ΔVEC} mice as indicated by normal ejection fraction and fractional shortening. n=5 control and n=9 Prox1^{ΔVEC} mice. Data is represented as mean±−SD. A indicates aorta. **P<0.01;*P<0.05; ****P<0.0001. Ctrl indicates control.

FIGS. 2A to 2D show that the loss of PROX1 (Prospero-related homeobox transcription factor 1) from aortic valvular endothelial cells (VECs) results in valve thickening, damaged endothelium, thrombus formation, and downregulation of FOXC2 (Forkhead box C2 transcription factor) in VECs.

FIG. 2A, Representative scanning electron microscope (SEM) images of the tri-cuspid aortic valve leaflets of Prox1f/f and Prox1ΔVEC mice. SEM images show that Prox1ΔVEC mice have thicker aortic valves, disrupted endothelial layer (arrows in the middle row) with infiltration of platelet-like cells (arrows in the bottom row). n=5 for the Prox1f/f and n=6 for Prox1ΔVEC mice. FIG. 2B, Representative Movat Pentachrome-stained histological images demonstrated thrombus-like structure in the aortic valve of a Prox1^{ΔVEC} mouse (arrow). The graph shows the quantification of the percentage of mice with thrombi in the aortic valves. Valves were analyzed by either SEM or Movat Pentachrome-staining of sections. n=10 control. mice (5 by SEM, 5 by staining) and n=16 Prox1ΔVEC mice (6 by SEM and 10 by staining). FIG. 2C, VWF (von Willebrand Factor) expression was increased in the downstream side VECs of Prox1^{ΔVEC} mice (arrows). n=5 for control mice; n=7 for Prox1^{ΔVEC} mice. FIG. 2D, FOXC2+VECs (arrows) were reduced in Prox1^{ΔVEC} mice. n=4 for control mice; n=5 for Prox1ΔVEC mice. Data are represented as mean±SEM. Each dot represents the average of 3-5 sections from a mouse. *P<0.05; ****P<0.0001. CD31 indicates cluster of differentiation 31; Ctrl, control; and DAPI, 4′,6-diamidino-2-phenylindole.

The ADAMTS (a disintegrin and metalloproteinase with thrombospondin motifs) family of proteases plays an important role in degrading versican and aggrecan.²¹ Among the 19 members of the family, ADAMTS-1, ADAMTS-4, ADAMTS-5, ADAMTS-9, ADAMTS-15, and ADAMTS-20 can cleave versican and aggrecan.²² Adamts-1, Adamts-4, Adamts-5, and Adamts-9 are downregulated in human myxomatous mitral valves,²² and Adamts-9^{+/-} and Adamts-5^{-/-} mice develop myxomatous heart valves.^{23,24} Mutations in ADAMTS-19 are associated with nonsyndromic progressive heart valve disease.²⁵ It was hypothesized that the elevated levels of aggrecan and versican in the valves of Prox1^{ΔVEC} mice arise from reduced ADAMTS activity. To test this hypothesis, the inventors used immunohistochemistry to detect a peptide fragment (DPEAAE) that is released by ADAMTS mediated cleavage of versican. Consistent with a previous report, the inventors observed high levels of cleaved versican (DPEAAE) in control valves (FIG. 3C).²⁴ In contrast, DPEAAE levels were significantly reduced in the aortic valves of 6-month-old Prox1^{ΔVEC} mice (FIG. 3C) and showed a trend toward reduction in the mitral valves of mutants. The inventors extracted RNA from aortic and mitral valves of 3-month-old control and Prox1^{ΔVEC} mice and found that Adamts-1, Adamts-5, and Adamts-9 transcript levels were reduced, albeit in a nonsignificant manner, in Prox1^{ΔVEC} valves (FIG. 3D).

Immunohistochemistry with collagen hybridizing peptide did not reveal significant changes in collagen expression in Prox1^{ΔVEC} mice compared with controls (FIG. 3E). However, collagen fibers appeared to be disrupted. Indeed, collagen fibers appeared to be disrupted in portions of the aortic valves of Prox1^{ΔVEC} mice in transmission electron microscopy images as well (FIG. 3F, arrows). Correspondingly, collagen fibers appeared to be absent from portions of the valve (FIG. 3F, asterisk). Elastin fibers, which are normally localized to the upstream side, were quantified as described by Gomez-Stallons et al.²⁶ The elastin fibers were disrupted and mislocalized to the downstream and interstitial layers in Prox1^{ΔVEC} valves (FIG. 3G).

The inventors did not observe Alizarin red-calcific nodules in the aortic valves of chow or high-fat diet fed Prox1^{ΔVEC} mice. Thus, Prox1^{ΔVEC} mice do not develop calcific aortic valve disease. Instead, deletion of Prox1 from VECs led to the progressive thickening of heart valves, disruption of VEC layer with platelet infiltration, aberrant accumulation of unprocessed proteoglycans, and disruption of collagen and elastin fibers. Based on the combined data, it was concluded that Prox1^{ΔVEC} valves undergo late-onset myxomatous degeneration.

FIGS. 3A to 3G show that increased proteoglycan expression and disrupted collagen and elastin fibers are observed in the aortic valves of Prox1^{ΔVEC} mice. FIG. 3A through FIG. 3C, Representative immunofluorescence images and semiquantitative measurements of aggrecan (FIG. 3A), versican (FIG. 3B), and cleaved versican (anti-DPEAAE peptide in FIG. 3C) expression in the aortic valve sections from 6-month-old control and Prox1ΔVEC mice. Aggrecan and versican were significantly increased and cleaved versican was significantly reduced in Prox1ΔVEC mice. Each dot in the graph represents the average of 3-5 sections from a mouse. n=8 control and n=13 Prox1^{ΔVEC} in A; n=6 control and n=10 Prox1^{ΔVEC} in B; n=7 control and n=8 Prox1^{ΔVEC} in FIG. 3C. FIG. 3D, Aortic and mitral valve tissues were pooled together from five 3-month-old control or 5 Prox1ΔVEC mice for generating 1 pooled sample. RNA was extracted from the pooled samples and quantitative real-time polymerase chain reaction (qRT-PCR) was performed to quantify the expression of selected genes. Each dot in the graph represents data from one pooled sample. The dotted line represents the mean expression in control samples. n=3 control and n=3 Prox1ΔVEC pooled samples. qRT-PCR showed that 3 out of the 4-candidate proteoglycan degrading enzymes had a trend towards reduced expression in Prox1^{ΔVEC} samples. FIG. 3E, Collagen binding peptide staining followed by semiquantitative measurement suggested comparable expression levels in the aortic valves of 6-month-old control and Prox1^{ΔVEC} mice. However, the collagen fibers in the aortic valves of Prox1^{ΔVEC} mice appeared to be disrupted (arrowhead). n=8 control and n=7 Prox1^{ΔVEC} mice (FIG. 3F) Representative transmission electron microscope (TEM) images show valvular endothelial cells (VECs) (dark band at the bottom of the image) and valvular interstitial cell-like cells (middle gray area) in the aortic valves of 12-month-old mice. Well-organized collagen fibers (arrows) were seen in control mice. In contrast, collagen fibers appeared to be disrupted in Prox1^{ΔVEC} mice (arrows) with gaps in between the bundles (asterisk). FIG. 3G, Resorcin-Fuchsin staining showed normal elastin expression in the upstream side of aortic valves (arrowhead left image, bottom arrow right image) in 6-month-old control mice. In contrast, normal elastin distribution was reduced and abnormal distribution within and in the downstream side of aortic valves (arrowhead) was observed in 6-month-old Prox1^{ΔVEC} mice. The graph shows normal and abnormal elastin expression in control and Prox1^{ΔVEC} mice at various ages. Data are represented as the percentage of total mice. 1-month-old mice: n=8 controls and n=9 Prox1ΔVEC; 3-month-old mice: n=7 controls and n=8 Prox1ΔVEC; 6-month-old mice: n=11 controls and Prox1^{ΔVEC}; 12-month-old mice: n=5 controls and n=8 Prox1^{ΔVEC} Data are represented as Mean±SEM in (A, B, C, and E). Data are represented as mean±SD in D. *P<0.05, ****P<0.0001. CD31 indicates cluster of differentiation 31; Ctrl, control; and DAPI, 4′,6-diamidino-2-phenylindole.

PDGF-B and FOXC2 Are Physiologically Relevant Targets of PROX1 in VECs.

VICs control ECM deposition and organization. Abnormal EndMT could result in an increase in VIC numbers and consequently ECM disorganization. The inventors determined that the VIC numbers were not increased and that their density was reduced in the aortic and mitral valves of Prox1^{ΔVEC} mice. The inventors also did not observe any obvious increase in the number of SMA (smooth muscle actin)⁺myofibroblasts (a marker of EndMT) in 6-month old Prox1^{ΔVEC} mice although a mild to moderate increase was observed in 12-month-old mutants. These observations suggested that the ECM composition of Prox1^{ΔVEC} mice is not defective due to an increase in the number of VICs or EndMT but is likely caused by an imbalance in ECM synthesis and degradation.

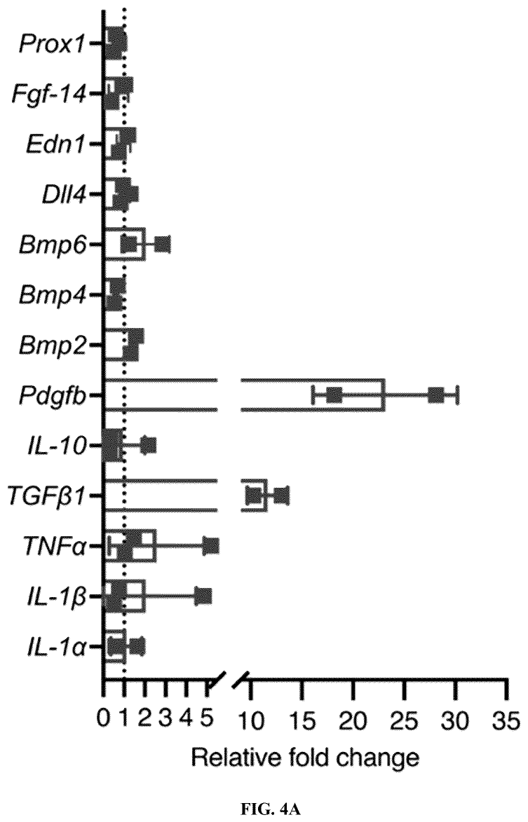

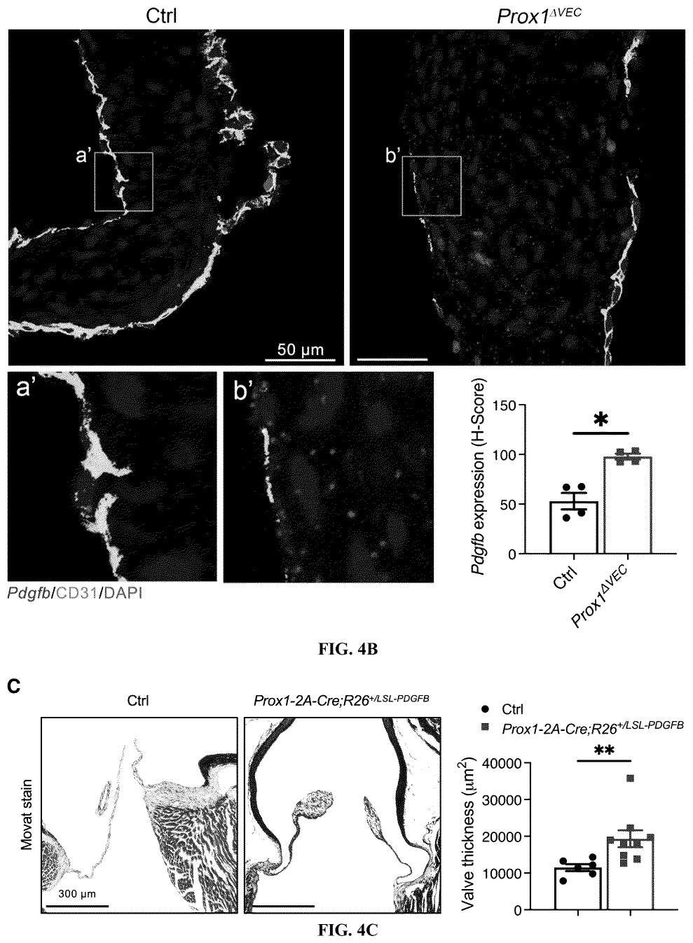

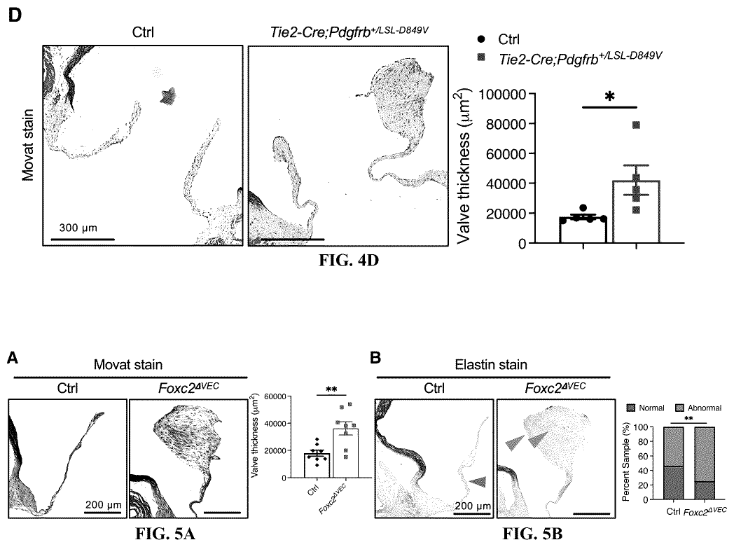

VECs are known to regulate VICs in a paracrine manner. To determine if signaling defects contribute to valve thickening in Prox1^{ΔVEC} mice, the inventors examined the abundance of transcripts encoding 12 common cytokines and growth factors. The inventors found that Pdgfb was increased 23.1-fold in Prox1^{ΔVEC} valves when compared with control samples and Tgfb1 was increased 11.6-fold (FIG. 4A), suggesting that PROX1 negatively regulates the expression of these genes in VECs. To test this hypothesis, the inventors used a PROX1-specific siRNA (small interfering RNA) in sheep mitral VECs and observed upregulation of PDGFB, but not TGFB1. Moreover, the inventors used the modified in situ hybridization approach RNAscope to validate the quantitative real-time polymerase chain reaction data for Pdgfb. The inventors observed a few Pdgfb⁺ puncta in control valves. In contrast, Pdgfb was strikingly upregulated in both VECs and VICs of 3-month-old Prox1^{ΔVEC} mice (FIG. 4B). These observations are consistent with the upregulation of Pdgfb in Prox1^{ΔVEC} VECs.

TGF-β1 is a known regulator of ECM composition, and mutations in genes that regulate TGF-β signaling are associated with syndromic mitral valve defects such as Marfan syndrome.²⁹ PDGF-B is also a potent regulator of ECM composition and Pdgfb^{-/-} mice that die perinatally possess hypoplastic heart valves.^{30,31} However, little is known about the role of PDGF-B in heart valve disease. The inventors bred the Prox1-2A-Cre and R26^{+/LSL-PDGFB} mice to overexpress PDGF-B in the VECs.³² The aortic and mitral valves of Prox12A-Cre and R26^{+/LSL-PDGFB} mice were significantly thicker than that of their littermates (FIG. 4C). This data suggested that the increased expression of PDGF-B in VECs is responsible for the thickening of Prox1^{ΔVEC} valves.

PDGFRβ (PDGF receptor β) is the cognate receptor of PDGF-B, and it is expressed in connective tissue cells such as VICs. de novo W566R or P584R mutations in PDGFRβ result in constitutive activation of the receptor and the rare disease Kosaki overgrowth syndrome.^{33,34} MVP was identified in a patient with the W566R mutation and another patient with the P584R mutation.^{33,35} To investigate the physiological significance of PDGF-B signaling in valve disease, the inventors used a previously reported model to conditionally express constitutively active PDGFRβ^{D849V} in VICs.³⁶ The D849V mutation is in the kinase domain of the receptor. The inventors bred the Pdgfrb^{+/LSLD849V} with Tie2-Cre to induce PDGFRβ^{D849V} expression specifically in the PDGFRβ⁺ cells of heart valves.¹² Analysis of 6-month-old mice revealed significantly thicker valves and more proteoglycan deposition (stained) in Tie2-Cre; Pdgfrb^{+/LSL-D849V} mice compared with control littermates (FIG. 4D). Thus, overexpression of PDGF-B in VECs or hyperactivation of PDGFRβ signaling in VICs can recapitulate the phenotype of Prox1^{ΔVEC} mice.

Together, these results show that PDGF-B is a physiologically relevant target of PROX1 and a novel regulator of myxomatous valve disease. PROX1 could directly regulate PDGF-B or indirectly through an intermediate transcription factor. As shown in FIG. 2D, FOXC2 expression is downregulated in the VECs of Prox1^{ΔVEC} mice. FOXC2 inhibits PDGF B expression in the lymphatic endothelial cells.^{37,38} Hence, the inventors tested if FOXC2 is a physiologically relevant target of PROX1 in VECs. Foxc2^{-/-} embryos die between E13.5 and birth. The inventors have developed a novel small hairpin RNA-based strategy to overcome the embryonic lethality of Foxc2^{-/-} mice and to knockdown Foxc2 in VECs.³⁹ The inventors combined Notch1Cre^{Lo} and CAG-LoxP-Stop-LoxP-rtTA3-mKate2 mice to express rtTA3 (reverse tetracycline transactivator 3) and the fluorescent marker mKate2 in VECs. The LoxP flanked transcriptional stop cassette (Stop) is removed by Cre recombinase resulting in the expression of rtTA3 and mKate2 from the CAG (cytomegalovirus (CMV) enhancer, beta-actin promoter, beta-globin splice acceptor) synthetic regulatory element. In the presence of doxycycline, rtTA3 binds and activates the TetO (tetracycline response element), inducing expression of GFP and a highly efficient, microRNA-based small hairpin RNA targeting Foxc2.

FIGS. 4A to 4D show that hyperactivation of PDGF-B (platelet-derived growth factor-B)/PDGFRβ (PDGF receptor β) signaling causes myxomatous degeneration of aortic valve. FIG. 4A, Aortic and mitral valve tissues were pooled together from five 3-month-old control or 5 Prox1^{ΔVEC} mice for generating 1 pooled sample. Following RNA extraction and cDNA synthesis from the pooled samples quantitative real-time polymerase chain reaction (qRT-PCR) was performed for the expression of selected cytokines and growth factors. Each dot represents data from 1 pooled sample. n=2 control and n=2 or 3 Prox1^{ΔVEC} pooled samples. The dotted line represents the mean expression in control samples. Data are represented as mean±SD. FIG. 4B, Representative RNAscope images for Pdgfb expression (red dots) in the aortic valves of 3-month-old control and Prox1ΔVEC mice. The lower parts are enlarged images of the boxed areas in the upper parts. Quantification of the RNAscope results showed that Prox1^{ΔVEC} mice had higher Pdgfb expression. Each dot represents the average from 3 sections of a mouse valve. n=4 mice per genotype. FIG. 4C, Representative Movat Pentachrome-stained images of aortic valves from 6-month-old control and Prox1-2A-Cre; R26^{+/LSL-PDGFB} mice. Thicker aortic valves were observed in Prox1-2A-Cre; R26^{+/LSL-PDGFB} mice when compared with control valves. n=6 control and n=9 Prox1-2A-Cre; R26^{+/LSL-PDGFB} mice. FIG. 4D, Representative Movat Pentachrome stain images of the aortic valves of 6-month-old control and Tie2-Cre; Pdgfrb^{+/D849V} mice. Tie2-Cre; Pdgfrb^{+/D849V} mice had enlarged aortic valves when compared with control valves. n=5 for control and Tie2-Cre; Pdgfrb^{+/D849V} mice. The area of valve leaflets was measured from 4 to 6 stained sections per valve. The average of these areas is represented as the thickness of valve in that mouse. *P<0.05. **P<0.01. CD31 indicates cluster of differentiation 31; Ctrl, control; and DAPI, 4′,6-diamidino-2-phenylindole.

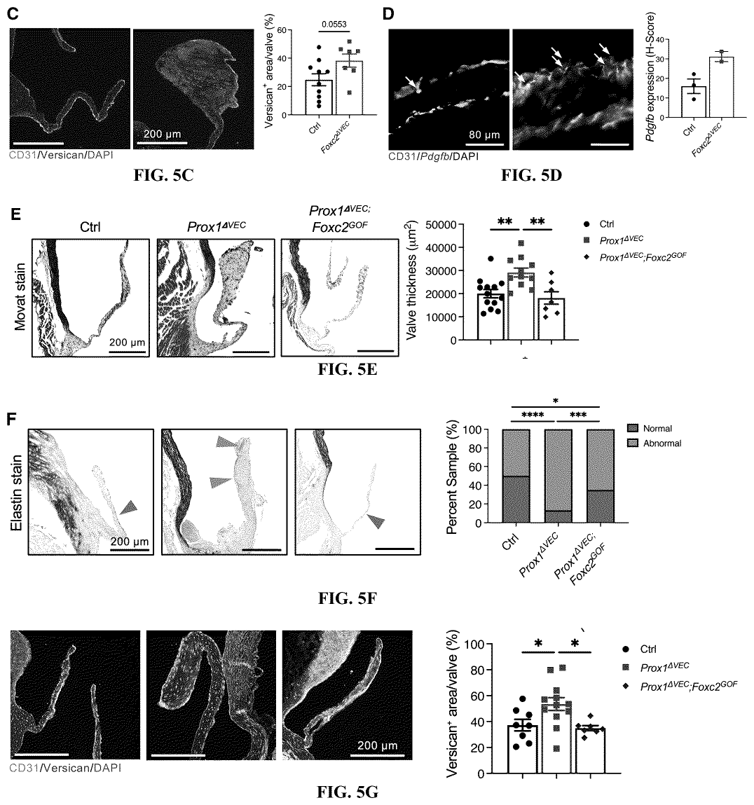

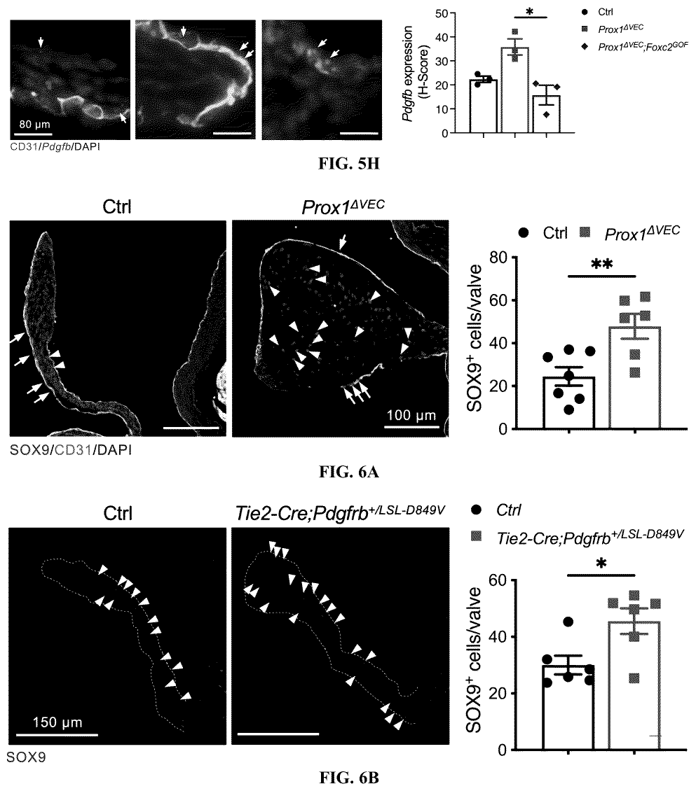

The inventors administered doxycycline to Notch1-Cre^{Lo}; CAG-LoxP-Stop-LoxP-rtTA3-mKate2; TetO-GFP-shFoxc2 mice (henceforth called as Foxc2^{ΔVEC}) from E10.5 and analyzed the heart valves at 12 months of age. The analysis revealed that the aortic valves of Foxc2^{ΔVEC} mice were significantly thicker than that of control littermates (FIG. 5A). Additionally, elastin expression was defective, the proteoglycan versican was enriched, and Pdgfb expression appeared to be increased in the aortic valves of Foxc2^{ΔVEC} mice (FIG. 5B through FIG. 5D).

The inventors previously reported a transgenic mouse model to overexpress FOXC2 in a Cre-dependent manner.¹⁰ In these FOXC2^{GOF} transgenic mice, FOXC2 cDNA was inserted downstream of the ubiquitously expressed CMV β-actin promoter and a LoxP-GFP-Stop-LoxP cassette.¹⁰ FOXC2 is expressed in cells that express Cre and their progeny.

By breeding FOXC2^{GOF} with Notch1-Cre^{Lo} FOXC2^{GOF}; Prox1^{ΔVEC} mice were generated to test whether restoration of FOXC2 expression could rescue the phenotype of Prox1^{ΔVEC} mice. Indeed, the thickness of the aortic valve is reduced in FOXC2^{GOF}; Prox1^{ΔVEC} compared with Prox1^{ΔVEC} mice (FIG. 5E). Furthermore, normal expression pattern of elastin was restored and the expression of versican and Pdgfb was normalized in the FOXC2^{GOF}; Prox1^{ΔVEC} mice (FIG. 5F through FIG. 5H).

The mitral valves of Foxc2^{ΔVEC} mice were not obviously defective, and restoration of FOXC2 expression was not sufficient to ameliorate the mitral valve defects of Prox1^{ΔVEC} mice. Together these data show that FOXC2 is a physiologically relevant target of PROX1 in aortic valves. Yet to be identified targets of PROX1 are likely compensating for the loss of FOXC2 in mitral valves.

FIGS. 5A to 5H shows that knockdown of FOXC2 (Forkhead box C2 transcription factor) results in enlarged aortic valves, and over-expression of FOXC2 rescues the aortic valve defects of Prox1^{ΔVEC} mice. FIG. 5A, Representative images of Movat Pentachrome staining that was performed using aortic valves of 12-month-old control and Foxc2^{ΔVEC} mice with graph showing the semiquantitative measurement of valve thickness. n=8 controls and n=8 Foxc2^{ΔVEC} mice. FIG. 5B, Representative images of Resorcin-Fuchsin staining that was performed using the aortic valves of 12-month-old control and Foxc2^{ΔVEC} mice. The arrowhead on the left image shows the normal expression of elastin on the upstream side of valves. The arrowheads in the right image show abnormal elastin expression within and in the downstream side of valves. The graph shows the distribution of normal and abnormal elastin expression in control and mutant mice. n=11 controls and n=7 Foxc2^{ΔVEC} mice. FIG. 5C, Representative immunofluorescence images for versican expression in the aortic valves of 12-month-old control and Foxc2^{ΔVEC} mice, followed by semiquantitative measurement. n=10 control and n=7 Foxc2^{ΔVEC} mice. FIG. 5D, Representative RNAscope images for Pdgfb expression (red dots) in the aortic valves of 6-month-old control and Foxc2ΔVEC mice (arrows indicate Pdgfb+ valvular endothelial cells [VECs]). Upon quantification, Pdgfb expression appeared to be increased in Foxc2^{ΔVEC} mice (mean H-score=16.0 in control versus 31.5 in Foxc2ΔVEC). Each dot represents the average of 3 sections from a mouse valve. n=3 control and n=2 Foxc2ΔVEC mice. FIG. 5E through FIG. 5H, Over expression of FOXC2 in Prox1^{ΔVEC} mice (Prox1^{ΔVEC}; Foxc2^{GOF}) ameliorates valve thickness (FIG. 5E), abnormal elastin distribution (FIG. 5F), increased proteoglycan expression (FIG. 5G) and Pdgfb expression (FIG. 5H). N=13 control, n=1 1 Prox1^{ΔVEC} and n=7 Prox1^{ΔVEC}; Foxc2^{ΔGOF} mice in (FIG. 5E); n=13 control, n=11 Prox1^{ΔVEC} and n=9 Prox1^{ΔVEC}; Foxc2^{ΔGOF} mice in (FIG. 5F); n=8 control, n=12 Prox1ΔVEC and n=7 Prox1^{ΔVEC}; Foxc2^{ΔGOF} mice in (FIG. 5G). n=3 for all 3 genotypes in (FIG. 5H). Mice were 6-month-old in (E-G) and 3-month-old in (H). Data are represented as mean±SEM. Each dot represents the average of 3 to 5 sections from a mouse. *P<0.05; **P<0.01; ***P<0.001; ****P<0.0001. Ctrl indicates control.

PDGF-B Upregulates Proteoglycan Expression in a SOX9-Dependent Manner.

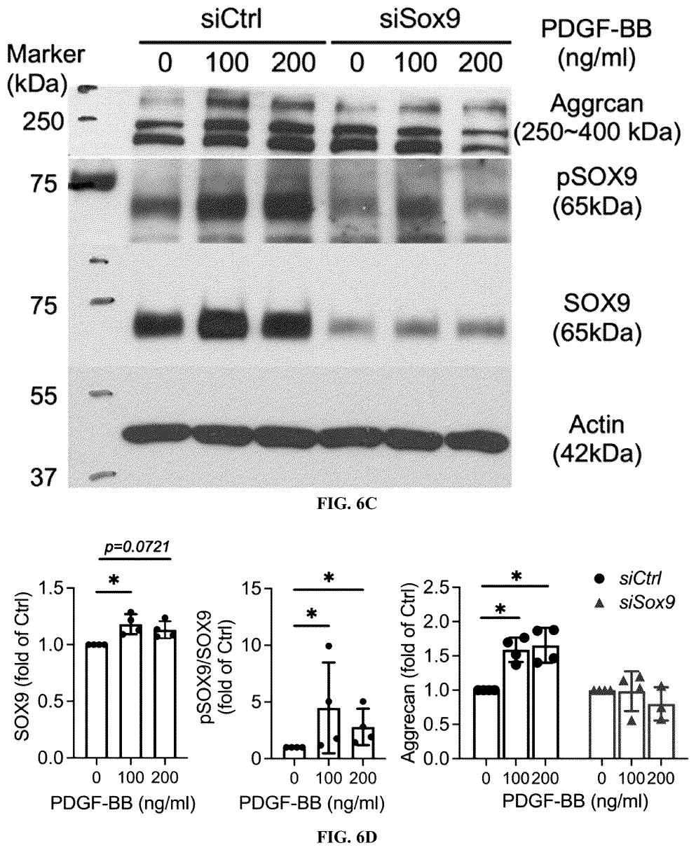

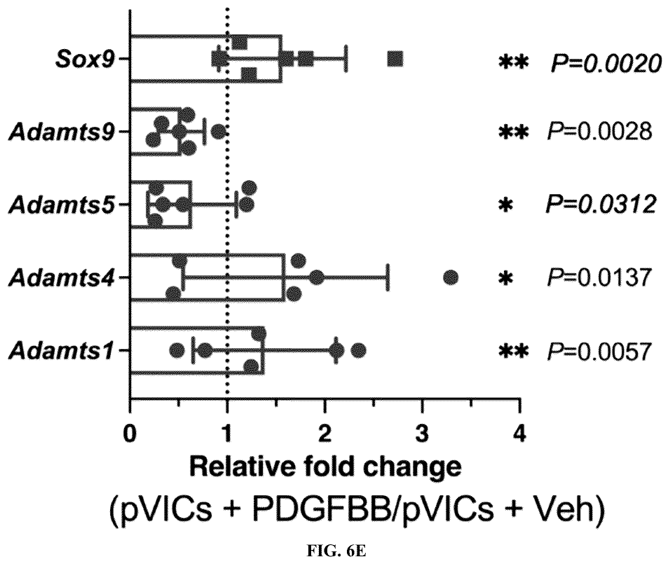

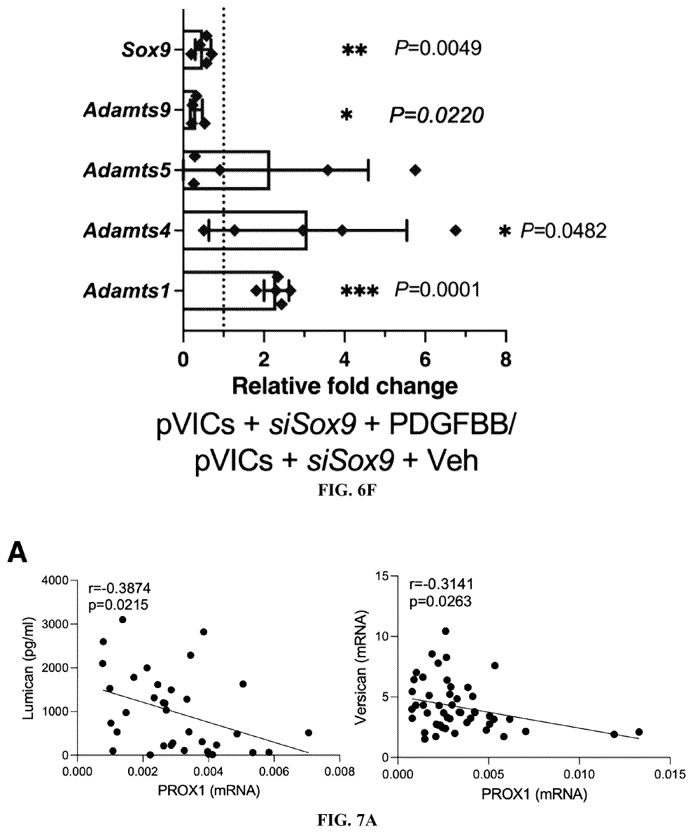

Valves share numerous similarities with connective tissue cell types such as chondrocytes,⁴⁰ and constitutive activation of PDGFRβ upregulates the transcription factor SOX9 (SRY-related HMG-box 9) in chondrocytes.³⁰ SOX9 is also upregulated in myxomatous valves from human and mice.^{41,42} Hence, the inventors asked if SOX9 is regulated by PROX1 and PDGF-B in heart valves. The inventors found that SOX9 was expressed in a few aortic VECs (FIG. 6A, arrows) and weakly in the VICs (FIG. 6A, arrowheads) of adult control mice as previously reported.⁴³ In contrast, SOX9 was strongly upregulated in the VICs of Prox1^{ΔVEC} mice (FIG. 6A). SOX9 expression was also significantly upregulated in the aortic VICs of 6-month-old Tie2-Cre; Pdgfrb^{+/LSL-D849V} mice (FIG. 6B). Consistent with these observations, SOX9 was also overexpressed in the mitral VICs of Prox1^{ΔVEC} and Tie2-Cre; Pdgfrb^{+/LSL-D849V} mice. Together, these findings are consistent with the hypothesis that PROX1 negatively regulates PDGFRB signaling and, in turn, SOX9 expression.

SOX9 activity is regulated by several post-translational mechanisms, which include phosphorylation.⁴³ To investigate the relationship between PDGF-B signaling, SOX9, and proteoglycan expression, the inventors treated porcine aortic valve VICs with the growth factor PDGF-B and observed increased SOX9, pSOX9 (phosphorylated SOX9), and aggrecan expression (FIG. 6C, FIG. 6D). Additionally, PDGF-B differentially regulated the expression of ADAMTS proteases in porcine aortic valve VICs: Adamts-9 and Adamts-5 were downregulated by PDGF-B treatment, whereas Adamts-4 and Adamts-1 were upregulated (FIG. 6E). Furthermore, PDGF-B-mediated changes in the levels of aggrecan and Adamts-5 in porcine aortic valve VICs were dependent on the expression of SOX9. Specifically, PDGF-B failed to upregulate aggrecan and downregulate Adamts-5 in siSOX9 (siRNA targeting SOX9)-transfected porcine aortic valve VICs (FIG. 6C, FIG. 6D, and FIG. 6F). These results suggest that PDGF-B signaling upregulates proteoglycan expression in a SOX9-dependent mechanism.

FIG. 6A to 6F show that PDGF-B (platelet-derived growth factor-B)/PDGFRβ (PDGF receptor β) signaling promotes proteoglycan expression in valvular interstitial cells (VICs) in a SOX9 (SRY-related HMG-box 9)-dependent manner. FIG. 6A, Representative immunohistochemistry images and quantification showing an increased number of SOX9+ cells in the aortic valves of 6-month-old Prox1^{ΔVEC} mice when compared with control littermates. Arrows indicate SOX9+ valvular endothelial cells (VECs) and arrowheads indicate SOX9+ VICs. n=7 control and n=6 Prox1^{ΔVEC} mice. FIG. 6B, SOX9 expression and quantification in the valves of 6-month-old control and Tie2-Cre; Pdgfrb^{+/D849V} mice. Arrowheads indicate SOX9+ VICs. n=7 for control and n=6 for Tie2-Cre; Pdgfrb^{+/D849V} mice. FIG. 6C, Representative Western blotting images using lysates prepared from porcine VICs (pVICs) transfected with siCtrl (control siRNA) or siSOX9 (siRNA targeting SOX9) and treated with the indicated amount of PDGFB for 24 hours. FIG. 6D, Quantification of the Western blotting data shows that PDGF-B increases the expressions of aggrecan, SOX9 (SRY-related HMG-box 9), and pSOX9 (phosphorylated SOX9) and that PDGF-B increases the expression of aggrecan in a SOX9-dependent manner. Protein expression was first normalized to actin (internal control) and then compared with expression in pVICs without PDGF-B treatment. Each dots represents an independent experiment. n=4 replicates. FIG. 6E and FIG. 6F, qRTPCR for the expressions of candidate ECM (extracellular matrix) degradation enzymes. RNA was collected from pVICs treated with vehicle or 100 ng/ml of PDGF-B for 24 hours in the presence (FIG. 6E) or absence (FIG. 6F) of siSox9 siRNA. Dotted line represents expression in vehicle-treated cells. Each dots represents an independent experiment. n=6 replicates in (FIG. 6E) and n=5 in (FIG. 6F). Each dot represents the average from 4 to 6 staining sections per mouse valve (FIG. 6B and FIG. 6D). Data is represented as mean±SEM in (A and B) and mean±SD in (FIG. 6E and FIG. 6F). Each dot represents an independent experiment. *P<0.05, **P<0.01, ***P<0.001. Veh indicates vehicle.

PDGFB and SOX9 Are Elevated in the Myxomatous Valves of Patients.

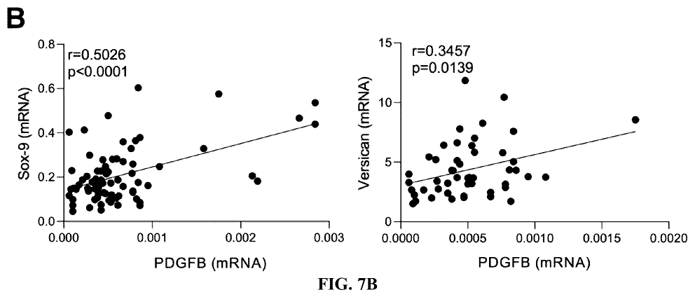

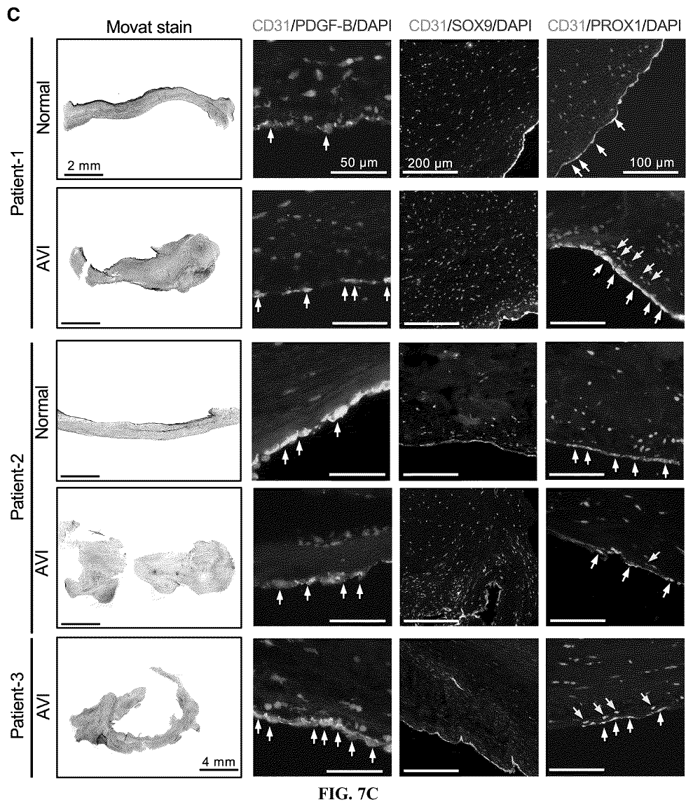

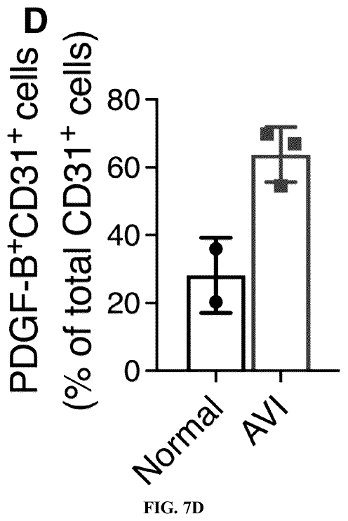

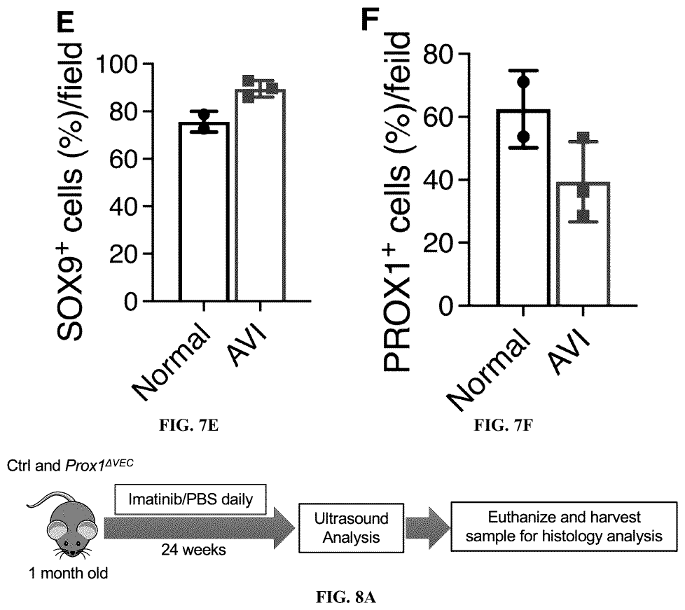

To investigate the clinical relevance of these findings, the inventors examined whether the phenotype of Prox1^{ΔVEC} mice recapitulates human valve disease. Myxomatous degeneration is the most common pathology associated with MVP in humans. Recently, the expression of VEC, VIC, and ECM molecules was evaluated in the valve tissue collected from patients with MVP.⁴⁴ Using these samples, it was found that the expression of the proteoglycans lumican and versican negatively correlated with PROX1 (FIG. 7A). Moreover, PDGFB positively correlated with SOX9 and versican (FIG. 7B).

The inventors analyzed aortic valve samples from 3 patients (5-year-old female, 7-year-old male, and 20-year-old male) with aortic valve stenosis and regurgitation. All patients had congenital aortic stenosis which was initially treated with balloon valvuloplasty. The patients subsequently developed insufficiency which required aortic valve replacement. Valve tissue from 2 patients had a normal-looking leaflet and a pathological leaflet. Valve tissue from the third patient did not have any normal-looking leaflet. All the leaflets were histologically analyzed.

The Hematoxylin & Eosin (H&E) and Movat Pentachrome-staining revealed that the pathological valve leaflets showed increased proteoglycan levels (DAPI) and increased thickness compared with the normal valve leaflet (FIG. 7C), consistent with myxomatous degeneration. Additionally, the pathological leaflets showed elevated PDGF-B expression and higher numbers of SOX9⁺ cells relative to the normal leaflet (FIG. 7C through FIG. 7E). However, the diseased aortic valve showed normal PROX1 expression in VECs (FIG. 7C, white arrows and FIG. 7F). Additionally, ectopic CD31⁻PROX1⁺ cells of unknown identity were observed in the diseased tissue (FIG. 7C, top arrows). These results suggest that PDGFB-PDGFRβ signaling is upregulated in myxomatous valves irrespective of the cause.

FIGS. 7A to 7F show that PDGF-B (platelet-derived growth factor-B) and SOX9 (SRY-related HMG-box 9) are increased in the myxomatous valves from human patients. FIG. 7A and FIG. 7B, Protein and RNA samples were obtained from the mitral valves of mitral valve prolapse patients (n=77) who underwent mitral valve replacement surgery. Genes and protein expressions were quantified by quantitative real-time polymerase chain reaction and ELISA respectively. Pearson correlation method was used for calculating r and p. FIG. 7C, Aortic valve leaflets from 3 patients with aortic valve insufficiency (AVI) were histologically analyzed. The relatively normal-looking leaflet was used as the internal control. The normal and pathological leaflets were analyzed using Movat Pentachrome stain and by immunohistochemistry for PDGF-B, SOX9, and PROX1. Bottom arrows indicate VECs and top arrows indicate PROX1+ cells of unknown identity in the valve interstitium. FIG. 7D through FIG. 7F, Expression of PDGF-B and SOX9 was increased in the diseased leaflet, but no obvious difference was observed in the number of PROX1+ cells. Data is represented as mean±SEM. Quantification was performed by averaging the data from 5 sections. PDGF-B staining was quantified as a percentage of PDGFB+CD31+ cells to total CD31+ cells. CD31+ indicates cluster of differentiation 31+; and PROX1, Prospero-related homeobox transcription factor 1.

Imatinib Attenuates Aortic Valve Degeneration in Prox1^{ΔVEC} Mice.

Imatinib is a Food and Drug Administration-approved drug that inhibits the activity of receptor tyrosine kinases (RTKs) such as PDGFRα, PDGFRβ, c-Kit (cellular counterpart of the viral transforming gene v-kit), and BCR-ABL (breakpoint cluster region-abelson).⁴⁵ Although imatinib is not specific for PDGFRβ, it has an excellent safety record in both humans and mice and it is used to treat leukemias and gastrointestinal cancer in humans. Most relevant to this work, imatinib ameliorated connective tissue disorders in patients with PDGFRβ hyperactivating mutations.³⁵

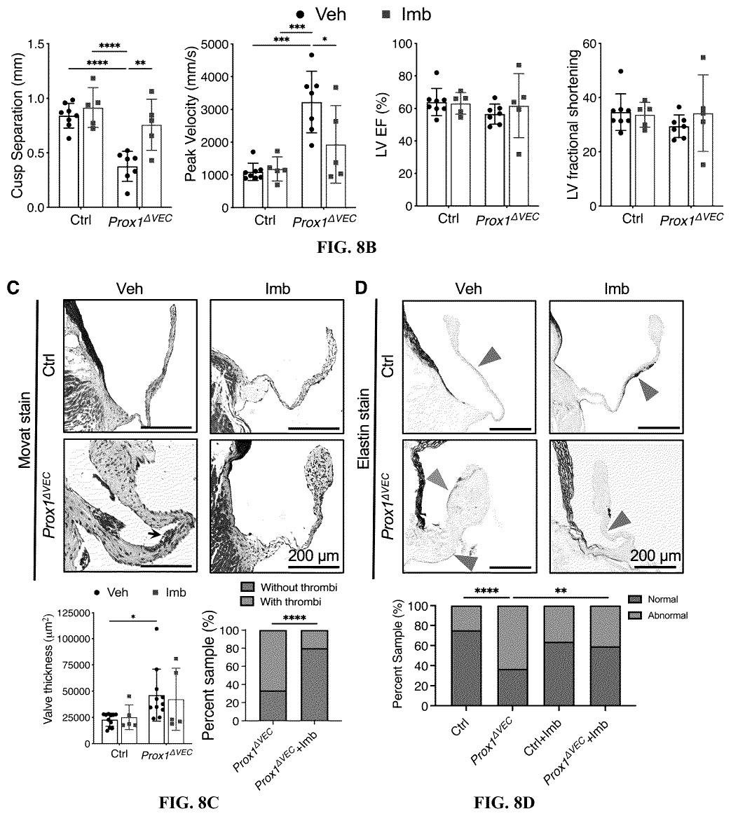

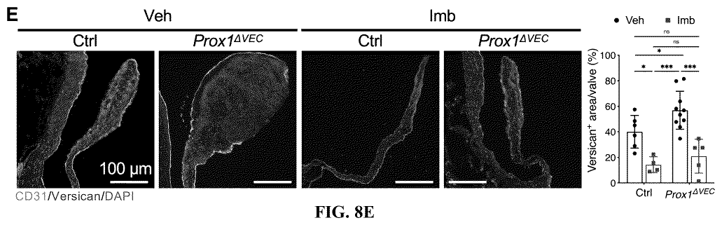

Given that these data show that loss of PROX1 in heart valves activates PDGF-B signaling, the inventors determined whether imatinib could be repurposed to inhibit the onset and progression of valve defects in Prox1^{ΔVEC} mice. To test this hypothesis, starting at postnatal day 30 the inventors orally administered imatinib to control and Prox1^{ΔVEC} mice daily at a dose of 50 mg/kg body weight for 6 months (FIG. 8A). Echocardiography showed that imatinib treatment preserves aortic valve function in Prox1^{ΔVEC} mice by maintaining cusp separation and preventing an increase in peak velocity (FIG. 8B). The inventors also observed platelet aggregate-like structures in untreated mutant aortic valves, which were reduced by imatinib treatment (FIG. 8C). Histological data suggested that imatinib treatment does not ameliorate the progressive thickening of Prox1^{ΔVEC} valves (FIG. 8C). However, imatinib significantly reduced the abnormal elastin expression on the downstream side of aortic valves in Prox1^{ΔVEC} mice (FIG. 8D, at the top of the bottom left image) while maintaining its expression on the upstream side (FIG. 8D, arrowheads in top images and bottom right image, bottom arrow in bottom left image). Imatinib also reduced the expression of versican in the aortic valves of Prox1^{ΔVEC} mice (FIG. 8E). Similarly, although imatinib did not reduce the thickness of the mitral valves it significantly normalized elastin and versican expression in Prox1^{ΔVEC} mice. Furthermore, imatinib normalized the expression of SOX9 in the aortic and mitral valves of Prox1^{ΔVEC} mice. These results reveal that imatinib treatment can partially rescue the valve defects of Prox1^{ΔVEC} mice by normalizing the expression of SOX9 and ECM components.

In summary, it is shown herein that PROX1 and FOXC2, molecules that are critical for the development of lymphatic vasculature and vascular valves, are necessary to prevent the myxomatous degeneration of heart valves.

FIG. 8A to 8E show that the receptor tyrosine kinase (RTK) inhibitor imatinib (Imb) partially rescues aortic valve function in Prox1^{ΔVEC} mice. FIG. 8A, Schematic of the experimental design. Imatinib (50 mg/kg-body weight/day) or PBS (vehicle [Veh]) was orally administrated to control and Prox1ΔVEC mice daily for 24 weeks starting from when they were 1-month-old. At the end of the treatment, aortic valves were evaluated by echocardiography and histology. FIG. 8B, Echocardiography showed that Imb enhanced cusp separation and lowered aortic peak velocity in Prox1^{ΔVEC} mice when compared with untreated Prox1^{ΔVEC} mice. There were no significant differences in left ventricle (LV) functions (ejection fraction and fractional shortening). n=7 Veh-treated controls, n=8 Veh-treated Prox1ΔVEC mice, n=5 Imb-treated controls and n=5 Imb-treated Prox1^{ΔVEC} mice. FIG. 8C, Representative Movat Pentachrome-stained sections from the aortic valves of control and Prox1^{ΔVEC} mice treated with Veh or Imb. The graphs show the quantification of valve thickness and the presence of thrombi. Arrow points to a thrombus-like structure in an untreated. Prox1^{ΔVEC} mouse. The number of thrombus-like structures was reduced in Prox1^{ΔVEC} mice treated with Imb. The area of valve leaflets was measured from 4 to 6 sections per valve. The average of these areas is represented as the thickness of valve in that mouse. Each dot represents an individual mouse. Valve thickness in Prox1^{ΔVEC} mice was not reduced by Imb treatment. n=12 Veh-treated controls, n=10 Veh-treated Prox1^{ΔVEC} mice, n=5 Imb-treated controls and n=5 Imb-treated Prox1^{ΔVEC} mice. FIG. 8D, Abnormal elastin distribution was rescued in the aortic valves of Imb-treated Prox1^{ΔVEC} mice. n=10 Veh-treated control, n=10 Veh-treated Prox1^{ΔVEC}, n=5 Imb-treated control and n=5 Imb-treated Prox1^{ΔVEC} mice. FIG. 8E, Immunohistochemistry and quantification indicated that abnormal versican production in Prox1^{ΔVEC} mice was inhibited by Imb treatment. n=6 Veh-treated controls, n=10 Veh-treated Prox1^{ΔVEC} mice, n=4 Imb-treated controls and n=5 Imb-treated Prox1^{ΔVEC} mice. Data are represented in Mean±SD in (B) and Mean±SEM in (C and E). Each dot represents the average of 3-5 sections from a mouse. *P<0.05; **P<0.01, ***P<0.001, ****P<0.0001.

As embodied and broadly described herein, an aspect of the present disclosure relates to a method for treating a patient suffering from myxomatous mitral valve disease (MMVD), the method comprising, consisting essentially of, or consisting of, administering to the patient an effective amount of a pharmaceutical composition comprising: a Prox1 gene, a Prox1 mimic, a Platelet Derived Growth Factor (PDGF) antagonist or a Platelet Derived Growth Factor Receptor Beta (PDGFRB) antagonist that prevents the thickening of heart valves and delays the onset of clinical symptoms of myxomatous valve disease in the patient. In one aspect, the PDGF or PDGFRB antagonist is selected from at least one of: AC710, AC710 Mesylate, AG1295, AG1296, an antagonistic human monoclonal or portion thereof targeting PDGFRB, an antagonistic human monoclonal or portion thereof targeting PDGF, avapritinib, axitinib, AZD2932, BOT-191, cediranib, celecoxib, CP 673451, crenolanib, dasatinib, Desethyl Sunitinib, DMPQ dihydrochloride, dovitinib, etoricoxib and DFU, ilorasertib, imatinib, imatinib mesylate, KG 5, lenvatinib, Linifanib, N-CP-673451, nilotinib, nintedanib, orantinib, pazopanib, PDGFRa kinase inhibitor-1, ponatibib, radotinib, regorafenib, ripretinib, sorafenib, SU 4312, SU 5402, SU14813, SU14813 maleate, SU16f, sunitinib, sunitinib malate, TAK 593, TAK-593, TG 100572, toceranib, or toceranib phosphate. In another aspect, the Prox1 gene or Prox1 mimic is an RNA, a DNA, or derivatives thereof. In another aspect, the PDGFRB antagonist is imatinib. In another aspect, the method further comprises adding one or more non-active pharmaceutically acceptable ingredients selected from at least one of: buffers, excipients, binders, diluents, vehicles, lubricants, wetting, emulsifying, salts, or carriers. In another aspect, the Prox1 gene, Prox1 mimic, PDGF antagonist, or PDGFRB antagonist is administered orally in the form of a tablet or a capsule. In another aspect, the administration of the Prox1 gene, Prox1 mimic, PDGF antagonist, or PDGFRB antagonist results in one or more of the following in the patient selected from the group consisting of: effects a prolongation of the preclinical phase without exhibiting clinical symptoms of heart failure, effects a delay of onset of clinical symptoms of heart failure, increases the survival time of the treated patient as compared to placebo treatment, improves the quality of life of the treated patient, improves cardiac function/output in the treated patient, reduces sudden cardiac death of the patient due to cardiac reasons, and reduces the risk of reaching heart failure. In another aspect, the patient is a mammal selected from the group consisting of: a human, a dog, a cat, and a horse. In another aspect, the Prox1 gene, Prox1 mimic, PDGF antagonist, or PDGFRB antagonist is administered in a daily dose of 0.1, 0.5, 1, 2, 3, 4, 5, 6, 7, 8, 9 10, 12.5, 15, 20, 25, 30, 40 50, 60, 75, 80, 90 or 100 mg/kg bodyweight. In another aspect, the daily dose of the Prox1 gene, Prox1 mimic, PDGF antagonist, or PDGFRB antagonist is administered as two doses 0.05, 0.5, 1, 2, 3, 4, 5, 6, 7, 8, 9 10, 12.5, 15, 20, 25, 30, 40, or 50 mg/kg bodyweight administered every 12 hours. In another aspect, the daily dose of the PDGF inhibitor imatinib is between 100, 200, 250, 300, 400, 500, 600, 700, 750, or 800 mg per day. In another aspect, the Prox1 gene, Prox1 mimic, PDGF antagonist, or PDGFRB antagonist is administered orally, intravenously, enterally, or parenterally. In another aspect, the administration of the Prox1 gene, Prox1 mimic, PDGF antagonist, or PDGFRB antagonist further effects a prolongation of the time of survival of the patient, as compared to placebo treatment or non-PDGFRB antagonist treatment, of at least about 30 days, at least about 5 months, or at least about 7 months.

As embodied and broadly described herein, an aspect of the present disclosure relates to a method for detecting and treating a patient suffering from myxomatous mitral valve disease (MMVD), the method comprising, consisting essentially of, or consisting of: identifying that the patient has at least one of: mutation or deletion of Prox1, platelet derived growth factor (PDGF) secretion is unregulated, or PDGF receptor beta (PDGFRB) signaling is unregulated; and administering to the patient an effective amount of a pharmaceutical composition comprising at least one of: a Prox1 gene, a Prox1 mimic, a PDGF antagonist, or a PDGFRB antagonist that prevents the thickening of heart valves and delays the onset of clinical symptoms of myxomatous valve disease in the patient. In one aspect, the PDGF or PDGFRB antagonist is selected from at least one of: AC710, AC710 Mesylate, AG1295, AG1296, an antagonistic human monoclonal or portion thereof targeting PDGFRB, an antagonistic human monoclonal or portion thereof targeting PDGF, avapritinib, axitinib, AZD2932, BOT-191, cediranib, celecoxib, CP 673451, crenolanib, dasatinib, Desethyl Sunitinib, DMPQ dihydrochloride, dovitinib, etoricoxib and DFU, ilorasertib, imatinib, imatinib mesylate, KG 5, lenvatinib, Linifanib, N-CP-673451, nilotinib, nintedanib, orantinib, pazopanib, PDGFRa kinase inhibitor-1, ponatibib, radotinib, regorafenib, ripretinib, sorafenib, SU 4312, SU 5402, SU14813, SU14813 maleate, SU16f, sunitinib, sunitinib malate, TAK 593, TAK-593, TG 100572, toceranib, or toceranib phosphate. In another aspect, the Prox1 gene or Prox1 mimic is an RNA, a DNA, or a derivative thereof. In another aspect, the PDGFRB antagonist is imatinib. In another aspect, the method further comprises adding one or more non-active pharmaceutically acceptable ingredients selected from at least one of: buffers, excipients, binders, diluents, vehicles, lubricants, wetting, emulsifying, salts, or carriers. In another aspect, the Prox1 gene, Prox1 mimic, PDGF antagonist, or PDGFRB antagonist is administered orally in the form of a tablet or a capsule. In another aspect, the administration of the Prox1 gene, Prox1 mimic, PDGF antagonist, or PDGFRB antagonist results in one or more of the following in the patient selected from the group consisting of: effects a prolongation of the preclinical phase without exhibiting clinical symptoms of heart failure, effects a delay of onset of clinical symptoms of heart failure, increases the survival time of the treated patient as compared to placebo treatment, improves the quality of life of the treated patient, improves cardiac function/output in the treated patient, reduces sudden cardiac death of the patient due to cardiac reasons, and reduces the risk of reaching heart failure. In another aspect, the patient is a mammal selected from the group consisting of: a human, a dog, a cat, and a horse. In another aspect, the Prox1 gene, Prox1 mimic, PDGF antagonist, or PDGFRB antagonist is administered in a daily dose of 0.1, 0.5, 1, 2, 3, 4, 5, 6, 7, 8, 9 10, 12.5, 15, 20, 25, 30, 40 50, 60, 75, 80, 90 or 100 mg/kg bodyweight. In another aspect, the daily dose of the Prox1 gene, Prox1 mimic, PDGF antagonist, or PDGFRB antagonist is administered as two doses 0.05, 0.5, 1, 2, 3, 4, 5, 6, 7, 8, 9 10, 12.5, 15, 20, 25, 30, 40, or 50 mg/kg bodyweight administered every 12 hours. In another aspect, the daily dose of the PDGF inhibitor imatinib is between 100, 200, 250, 300, 400, 500, 600, 700, 750, or 800 mg per day. In another aspect, the Prox1 gene, Prox1 mimic, PDGF antagonist, or PDGFRB antagonist is administered orally, intravenously, enterally, or parenterally. In another aspect, the administration of the Prox1 gene, Prox1 mimic, PDGF antagonist, or PDGFRB antagonist further effects a prolongation of the time of survival of the patient, as compared to placebo treatment or non-PDGFRB antagonist treatment, of at least about 30 days, at least about 5 months, or at least about 7 months.

As embodied and broadly described herein, an aspect of the present disclosure relates to a method for preventing suffering from myxomatous mitral valve disease (MMVD) in a human patient, the method comprising, consisting essentially of, or consisting of, administering to the human patient an effective amount of a pharmaceutical composition comprising a Platelet Derived Growth Factor (PDGF) antagonist or a Platelet Derived Growth Factor Receptor Beta (PDGFRB) antagonist that prevents the thickening of heart valves and delays the onset of clinical symptoms of myxomatous valve disease in the patient.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

For each of the claims, each dependent claim can depend both from the independent claim and from each of the prior dependent claims for each and every claim so long as the prior claim provides a proper antecedent basis for a claim term or element.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Nkomo V T, Gardin J M, Skelton T N, Gottdiener J S, Scott C G, Enriquez-Sarano M. Burden of valvular heart diseases: a population-based study. Lancet. 2006; 368:1005-1011. doi: 10.1016/S0140-6736(06)69208-8
2. Kodigepalli K M, Thatcher K, West T, Howsmon D P, Schoen F J, Sacks M S, Breuer C K, Lincoln J. Biology and biomechanics of the heart valve extracellular matrix. J Cardiovasc Dev Dis. 2020; 7:57. doi: 10.3390/jcdd7040057
3. Huk D J, Austin B F, Horne T E, Hinton R B, Ray W C, Heistad D D, Lincoln J. Valve endothelial cell-derived Tgfbeta1 signaling promotes nuclear localization of Sox9 in interstitial cells associated with attenuated calcification. Arterioscler Thromb Vasc Biol. 2016; 36:328-338. doi: 10.1161/ATVBAHA.115.306091
4. Goddard L M, Duchemin A L, Ramalingan H, Wu B, Chen M, Bamezai S, Yang J, Li L, Morley M P, Wang T, et al. Hemodynamic forces sculpt developing heart valves through a KLF2-WNT9B paracrine signaling axis. Dev Cell. 2017; 43:274-289.e5. doi: 10.1016/j.devcel.2017.09.023
5. Bosse K, Hans C P, Zhao N, Koenig S N, Huang N, Guggilam A, LaHaye S, Tao G, Lucchesi P A, Lincoln J, et al. Endothelial nitric oxide signaling regulates Notch1 in aortic valve disease. J Mol Cell Cardiol. 2013; 60:27-35. doi: 10.1016/j.yjmcc.2013.04.001
6. Simmons C A, Grant G R, Manduchi E, Davies P F. Spatial heterogeneity of endothelial phenotypes correlates with side-specific vulnerability to calcification in normal porcine aortic valves. Circ Res. 2005; 96:792-799. doi: 10.1161/01.RES.0000161998.92009.64
7. Geng X, Cha B, Mahamud M R, Lim K C, Silasi-Mansat R, Uddin M K, Miura N, Xia L, Simon A M, Engel J D, et al. Multiple mouse models of primary lymphedema exhibit distinct defects in lymphovenous valve development. Dev Biol. 2016; 409:218-233. doi: 10.1016/j.ydbio.2015.10.022
8. Srinivasan R S, Oliver G. Prox1 dosage controls the number of lymphatic endothelial cell progenitors and the formation of the lymphovenous valves. Genes Dev. 2011; 25:2187-2197. doi: 10.1101/gad.16974811
9. Hulin A, Hortells L, Gomez-Stallons M V, O'Donnell A, Chetal K, Adam M, Lancellotti P, Oury C, Potter S S, Salomonis N, et al. Maturation of heart valve cell populations during postnatal remodeling. Development. 2019; 146:dev173047. doi: 10.1242/dev.173047
10. Cha B, Geng X, Mahamud M R, FuJ, Mukherjee A, Kim Y, Jho E H, Kim T H, Kahn M L, Xia L, et al. Mechanotransduction activates canonical Wnt/beta-catenin signaling to promote lymphatic vascular patterning and the development of lymphatic and lymphovenous valves. Genes Dev. 2016; 30:1454-1469. doi: 10.1101/gad.282400.116
11. Cha B, Geng X, Mahamud M R, Zhang J Y, Chen L, Kim W, Jho E H, Kim Y, Choi D, Dixon J B, et al. Complementary Wnt sources regulate lymphatic vascular development via PROX1-Dependent Wnt/beta-Catenin Signaling. Cell Rep. 2018; 25:571-584.e5. doi: 10.1016/j.celrep.2018.09.049
12. Kisanuki Y Y, Hammer R E, Miyazaki J, Williams S C, Richardson J A, Yanagisawa M. Tie2-Cre transgenic mice: a new model for endothelial cell-lineage analysis in vivo. Dev Biol. 2001; 230:230-242. doi: 10.1006/dbio.2000.0106
13. Iwano T, Masuda A, Kiyonari H, Enomoto H, Matsuzaki F. Prox1 postmitotically defines dentate gyrus cells by specifying granule cell identity over CA3 pyramidal cell fate in the hippocampus. Development. 2012; 139:3051-3062. doi: 10.1242/dev.080002
14. Wu B, Baldwin H S, Zhou B. Nfatc1 directs the endocardial progenitor cells to make heart valve primordium. Trends Cardiovasc Med. 2013; 23:294-300. doi: 10.1016/j.tcm.2013.04.003
15. Vooijs M, Ong C T, Hadland B, Huppert S, Liu Z, Korving J, van den Born M, Stappenbeck T, Wu Y, Clevers H, et al. Mapping the consequence of Notch1 proteolysis in vivo with NIP-CRE. Development. 2007; 134:535-544. doi: 10.1242/dev.02733
16. Garg V, Muth A N, Ransom J F, Schluterman M K, Barnes R, King I N, Grossfeld P D, Srivastava D. Mutations in NOTCH1 cause aortic valve disease. Nature. 2005; 437:270-274. doi: 10.1038/nature03940
17. Theodoris C V, Mourkioti F, Huang Y, Ranade S S, Liu L, Blau H M, Srivastava D. Long telomeres protect against age-dependent cardiac disease caused by NOTCH1 haploinsufficiency. J Clin Invest. 2017; 127:1683-1688. doi: 10.1172/JC190338
18. Theodoris C V, Zhou P, Liu L, Zhang Y, Nishino T, Huang Y, Kostina A, Ranade S S, Gifford C A, Uspenskiy V, et al. Network-based screen in iPSCderived cells reveals therapeutic candidate for heart valve disease. Science. 2021; 371:eabd0724. doi: 10.1126/science.abd0724
19. Nus M, MacGrogan D, Martinez-Poveda B, Benito Y, Casanova J C, Fernandez-Aviles F, Bermejo J, de la Pompa J L. Diet-induced aortic valve disease in mice haploinsufficient for the Notch pathway effector RBPJK/CSL. Arterioscler Thromb Vase Biol. 2011; 31:1580-1588. doi: 10.1161/ATVBAHA.111.227561
20. Welsh J D, Hoofnagle M H, Bamezai S, Oxendine M, Lim L, Hall J D, Yang J, Schultz S, Engel J D, Kume T, et al. Hemodynamic regulation of perivalvular endothelial gene expression prevents deep venous thrombosis. J Clin Invest. 2019; 129:5489-5500. doi: 10.1172/JCI124791
21. Apte S S. ADAMTS Proteins: concepts, challenges, and prospects. Methods Mol Biol. 2020; 2043:1-12. doi: 10.1007/978-1-4939-9698-8_1
22. Hulin A, Deroanne C F, Lambert C A, Dumont B, Castronovo V, Defraigne J O, Nusgens B V, Radermecker M A, Colige A C. Metallothionein-dependent upregulation of TGF-beta2 participates in the remodelling of the myxomatous mitral valve. Cardiovasc Res. 2012; 93:480-489. doi: 10.1093/cvr/cvr337
23. Kern C B, Wessels A, McGarity J, Dixon L J, Alston E, ArgravesWS, Geeting D, Nelson C M, Menick D R, Apte S S. Reduced versican cleavage due to Adamts9 haploinsufficiency is associated with cardiac and aortic anomalies. Matrix Biol. 2010; 29:304-316. doi: 10.1016/j.matbio.2010.01.005
24. Dupuis L E, McCulloch D R, McGarity J D, Bahan A, Wessels A, Weber D, Diminich A M, Nelson C M, Apte S S, Kern C B. Altered versican cleavage in ADAMTS5 deficient mice; a novel etiology of myxomatous valve disease. Dev Biol. 2011; 357:152-164. doi: 10.1016/j.ydbio.2011.06.041
25. Wunnemann F, Ta-Shma A, Preuss C, Leclerc S, van Vliet P P, Oneglia A, Thibeault M, Nordquist E, Lincoln J, Scharfenberg F, et al; MIBAVA Leducq Consortium principal investigators. Loss of ADAMTS19 causes progressive non-syndromic heart valve disease. Nat Genet. 2020; 52:40-47. doi: 10.1038/s41588-019-0536-2
26. Gomez-Stallons M V, Tretter J T, Hassel K, Gonzalez-Ramos O, Amofa D, Ollberding N J, Mazur W, Choo J K, Smith J M, Kereiakes D J, et al. Calcification andextracellular matrix dysregulation in human postmortem andsurgicalaortic valves. Heart. 2019; 105:1616-1621. doi: 10.1136/heartjnl-2019-314879
27. Abdelazeem B, Hollander R M, Gresham T M, Gjeka R, Kunadi A. Aortic valve insufficiency due to myxomatous degeneration: a case report and literature review. AME Case Rep. 2022; 6:10. doi: 10.21037/acr-21-68
28. Rabkin E, Aikawa M, Stone J R, Fukumoto Y, Libby P, Schoen F J. Activated interstitial myofibroblasts express catabolic enzymes and mediate matrix remodeling in myxomatous heart valves. Circulation. 2001; 104:2525-2532. doi: 10.1161/hc4601.099489
29. Ng C M, Cheng A, Myers L A, Martinez-Murillo F, Jie C, Bedja D, Gabrielson K L, Hausladen J M, Mecham R P, Judge D P, et al. TGF-beta-dependent pathogenesis of mitral valve prolapse in a mouse model of Marfan syndrome. J Clin Invest. 2004; 114:1586-1592. doi: 10.1172/JC122715
30. Kwon H R, Kim J H, Woods J P, Olson L E. Skeletalstemcellfatedefects caused by Pdgfrb activating mutation. Development. 2021; 148:dev199607. doi: 10.1242/dev.199607
31. Van den Akker N M, Winkel L C, Nisancioglu M H, Maas S, Wisse L J, Armulik A, Poelmann R E, Lie-Venema H, Betsholtz C, Gittenberger-de Groot A C. PDGFB signaling is important for murine cardiac development: its role in developing atrioventricular valves, coronaries, and cardiac innervation. Dev Dyn. 2008; 237:494-503. doi: 10.1002/dvdy.21436
32. Armulik A, Genove G, Mae M, Nisancioglu M H, Wallgard E, Niaudet C, He L, Norlin J, Lindblom P, Strittmatter K, et al. Pericytes regulate the blood-brain barrier. Nature. 2010; 468:557-561. doi: 10.1038/nature09522
33. Takenouchi T, Okuno H, Kosaki K. Kosaki overgrowth syndrome: A newly identified entity caused by pathogenic variants in platelet-derived growth factor receptor-beta. Am J Med Genet C Semin Med Genet. 2019; 181:650-657. doi: 10.1002/ajmg.c.31755
34. Takenouchi T, Yamaguchi Y, Tanikawa A, Kosaki R, Okano H, Kosaki K. Novel overgrowth syndrome phenotype due to recurrent de novo PDGFRB mutation. J Pediatr. 2015; 166:483-486. doi: 10.1016/j.jpeds.2014.10.015
35. Wenger T L, Bly R A, Wu N, Albert C M, Park J, Shieh J, Chenbhanich J, Heike C L, Adam M P, Chang I, et al. Activating variants in PDGFRB resultin a spectrum of disorders responsive to imatinib monotherapy. Am J Med Genet A. 2020; 182:1576-1591. doi: 10.1002/ajmg.a.61615
36. Olson L E, Soriano P. PDGFRbeta signaling regulates mural cell plasticity and inhibits fat development. Dev Cell. 2011; 20:815-826. doi: 10.1016/j.devcel.2011.04.019
37. Petrova T V, Karpanen T, Norrmen C, Mellor R, Tamakoshi T, Finegold D, Ferrell R, Kerjaschki D, Mortimer P, Yla-Herttuala S, et al. Defective valves and abnormal mural cell recruitment underlie lymphatic vascular failure in lymphedema distichiasis. Nat Med. 2004; 10:974-981. doi: 10.1038/nm1094
38. Gonzalez-Loyola A, Bovay E, Kim J, Lozano T W, SabineA, Renevey F, Arroz-Madeira S, Rapin A, Wypych T P, Rota G, et al. FOXC2 controls adult lymphatic endothelial specialization, function, and gut lymphatic barrier preventing multiorgan failure. Sci Adv. 2021; 7:eabf4335. doi: 10.1126/sciadv.abf4335
39. Premsrirut P K, Dow L E, Kim S Y, Camiolo M, Malone C D, Miething C, Scuoppo C, Zuber J, Dickins R A, Kogan S C, et al. A rapid and scalable system for studying gene function in mice using conditional RNA interference. Cell. 2011; 145:145-158. doi: 10.1016/j.cell.2011.03.012
40. Lincoln J, Lange A W, Yutzey K E. Hearts and bones: shared regulatory mechanisms in heart valve, cartilage, tendon, and bone development. Dev Biol. 2006; 294:292-302. doi: 10.1016/j.ydbio.2006.03.027
41. Cheek J D, Wirrig E E, Alfieri C M, James J F, Yutzey K E. Differential activation of valvulogenic, chondrogenic, and osteogenic pathways in mouse models of myxomatous and calcific aortic valve disease. J Mol Cell Cardiol. 2012; 52:689-700. doi: 10.1016/j.yjmcc.2011.12.013
42. Sainger R, Grau J B, Branchetti E, Poggio P, Seefried W F, Field B C, Acker M A, Gorman R C, Gorman J H 3rd, Hargrove C W 3rd, et al. Human myxomatous mitral valve prolapse: role of bone morphogenetic protein 4 in valvular interstitial cell activation. J Cell Physiol. 2012; 227:2595-2604. doi: 10.1002/jcp.22999
43. Gallina D, Lincoln J. Dynamic expression profiles of Sox9 in embryonic, post natal, and adult heart valve cell populations. Anat Rec (Hoboken). 2019; 302:108-116. doi: 10.1002/ar.23913
44. Ibarrola J, Garcia-Pena A, Matilla L, Bonnard B, Sadaba R, Arrieta V, Alvarez V, Fernandez-Celis A, Gainza A, Navarro A, et al. A new role for the aldosterone/mineralocorticoid receptor pathway in the development of mitral valve prolapse. Circ Res. 2020; 127:e80-e93. doi: 10.1161/CIRCRESAHA.119.316427
45. Fabian M A, Biggs W H 3rd, Treiber D K, Atteridge C E, Azimioara M D, Benedetti M G, Carter T A, Ciceri P, Edeen P T, Floyd M, et al. A small molecule-kinase interaction map for clinical kinase inhibitors. Nat Biotechnol. 2005; 23:329-336. doi: 10.1038/nbt1068
46. Delling F N, Vasan R S. Epidemiologyandpathophysiologyofmitralvalve prolapse: new insights into diseaseprogression, genet-ics, and molecular basis. Circulation. 2014; 129:2158-2170. doi: 10.1161/CIRCULATIONAHA.113.006702
47. Wang Y, Jin Y, Mae M A, Zhang Y, Ortsater H, Betsholtz C, Makinen T, Jakobsson L. Smooth muscle cell recruitment to lymphatic vessels requires PDGFB and impacts vessel size but not identity. Development. 2017; 144:3590-3601. doi: 10.1242/dev.147967
48. Johnson N C, Dillard M E, Baluk P, McDonald D M, Harvey N L, Frase S L, Oliver G. Lymphatic endothelial cell identity is reversible and its maintenance requires Prox1 activity. Genes Dev. 2008; 22:3282-3291. doi: 10.1101/gad.1727208
49. Silver B J, Jaffer F E, Abboud H E. Platelet-derived growth factor synthesis in mesangial cells: induction by multiple peptide mitogens. Proc Natl Acad Sci USA. 1989; 86:1056-1060. doi: 10.1073/pnas.86.3.1056
50. Sabuda-Widemann D, Grabensee B, Schwandt C, Blume C. Mycopheno-lic acid inhibits the autocrine PDGF-B synthesis and PDGF-BB-induced mRNA expression of Egr-1 in rat mesangial cells. Nephrol Dial Transplant. 2009; 24:52-61. doi: 10.1093/ndt/gfn462
51. Resnick N, Collins T, Atkinson W, Bonthron D T, Dewey C F Jr, Gimbrone M A Jr. Platelet-derived growth factor B chain promoter contains a cis-acting fluid shear-stress-responsive element. Proc Natl Acad Sci USA. 1993; 90:4591-4595. doi: 10.1073/pnas.90.10.4591
52. Sabine A, Agalarov Y, Maby-El Hajjami H, Jaquet M, Hagerling R, Pollmann C, Bebber D, Pfenniger A, Miura N, Dormond O, et al. Mechanotransduction, PROX1, and FOXC2 cooperate to control connexin37 and calcineurin during lymphatic-valve formation. Dev Cell. 2012; 22:430-445. doi: 10.1016/j.devcel.2011.12.020
53. Moore K, Fulmer D, Guo L, Koren N, Glover J, Moore R, Gensemer C, Beck T, Morningstar J, Stairley R, et al. PDGFRalpha: Expression and Function during Mitral Valve Morphogenesis. J Cardiovasc Dev Dis. 2021; 8:28-41. doi: 10.3390/jcdd8030028
54. Srinivasan R S, Geng X, Yang Y, Wang Y, Mukatira S, Studer M, Porto M P, Lagutin O, Oliver G. The nuclear hormone receptor Coup-TFII is required for the initiation and early maintenance of Prox1 expression in lymphatic endothelial cells. Genes Dev. 2010; 24:696-707. doi: 10.1101/gad.1859310
55. Geng X, Srinivasan S R. Whole-mount Immunohisto chemistry to Visualize Mouse Embryonic Dermal Lymphatic Vasculature. Bio Protoc. 2021; 11:e4186. doi: 10.21769/BioProtoc.4186
56. Varshney R, Murphy B, Woolington S, Ghafoory S, Chen S, Robison T, Ahamed J. Inactivation of platelet-derived TGF-beta1 attenuates aortic stenosis progression in a robust murine model. Blood Adv. 2019; 3:777-788. doi: 10.1182/bloodadvances.2018025817
57. Geng X, Srinivasan R S. Correlative fluorescence and scanning electron microscopy to study lymphovenous valve development. Methods Mol Biol. 2018; 1846:85-96. doi: 10.1007/978-1-4939-8712-2_6
58. Wylie-Sears J, Aikawa E, Levine R A, Yang J H, Bischoff J. Mitral valve endothelial cells with osteogenic differentiation potential. Arterioscler Thromb Vasc Biol. 2011; 31:598-607. doi: 10.1161/ATVBAHA.110.216184
59. Gould R A, Butcher J T. Isolation of valvular endothelial cells. J Vis Exp. 2010; 46:2158-2162. doi: 10.3791/2158.

What is claimed is:

1. A method for treating a patient suffering from myxomatous mitral valve disease (MMVD), the method comprising administering to the patient an effective amount of a pharmaceutical composition comprising: a Prox1 gene, a Prox1 mimic, a Platelet Derived Growth Factor (PDGF) antagonist or a Platelet Derived Growth Factor Receptor Beta (PDGFRB) antagonist that prevents thickening of heart valves and delays an onset of clinical symptoms of myxomatous valve disease in the patient.

2. The method of claim 1, wherein the PDGF or PDGFRB antagonist is selected from at least one of: AC710, AC710 Mesylate, AG1295, AG1296, an antagonistic human monoclonal or portion thereof targeting PDGFRB, an antagonistic human monoclonal or portion thereof targeting PDGF, avapritinib, axitinib, AZD2932, BOT-191, cediranib, celecoxib, CP 673451, crenolanib, dasatinib, Desethyl Sunitinib, DMPQ dihydrochloride, dovitinib, etoricoxib and DFU, ilorasertib, imatinib, imatinib mesylate, KG 5, lenvatinib, Linifanib, N-CP-673451, nilotinib, nintedanib, orantinib, pazopanib, PDGFRa kinase inhibitor-1, ponatibib, radotinib, regorafenib, ripretinib, sorafenib, SU 4312, SU 5402, SU14813, SU14813 maleate, SU16f, sunitinib, sunitinib malate, TAK 593, TAK-593, TG 100572, toceranib, or toceranib phosphate.

3. The method of claim 1, wherein the Prox1 gene or Prox1 mimic is an RNA, a DNA, or derivatives thereof.

4. The method of claim 1, wherein the PDGFRB antagonist is imatinib.

5. The method of claim 1, further comprising adding one or more non-active pharmaceutically acceptable ingredients selected from at least one of: buffers, excipients, binders, diluents, vehicles, lubricants, wetting, emulsifying, salts, or carriers.

6. The method of claim 1, wherein the Prox1 gene, Prox1 mimic, PDGF antagonist, or PDGFRB antagonist is administered orally as a tablet or a capsule.

7. The method of claim 1, wherein administration of the Prox1 gene, Prox1 mimic, PDGF antagonist, or PDGFRB antagonist results in one or more of the following in the patient selected from the group consisting of: effects a prolongation of a preclinical phase without exhibiting clinical symptoms of heart failure, effects a delay of onset of clinical symptoms of heart failure, increases a survival time of the treated patient as compared to placebo treatment, improves a quality of life of the treated patient, improves cardiac function/output in the treated patient, reduces sudden cardiac death of the patient due to cardiac reasons, and reduces a risk of reaching heart failure.

8. The method claim 1, wherein the patient is a mammal selected from the group consisting of: a human, a dog, a cat, and a horse.

9. The method of claim 1, wherein the Prox1 gene, Prox1 mimic, PDGF antagonist, or PDGFRB antagonist is administered in a daily dose of 0.1, 0.5, 1, 2, 3, 4, 5, 6, 7, 8, 9 10, 12.5, 15, 20, 25, 30, 40 50, 60, 75, 80, 90 or 100 mg/kg body weight.

10. The method of claim 1, wherein a daily dose of the Prox1 gene, Prox1 mimic, PDGF antagonist, or PDGFRB antagonist is administered as two doses 0.05, 0.5, 1, 2, 3, 4, 5, 6, 7, 8, 9 10, 12.5, 15, 20, 25, 30, 40, or 50 mg/kg body weight administered every 12 hours.

11. The method of claim 1, wherein a daily dose of PDGF inhibitor imatinib is between 100, 200, 250, 300, 400, 500, 600, 700, 750, or 800 mg per day.

12. The method of claim 1, wherein the Prox1 gene, Prox1 mimic, PDGF antagonist, or PDGFRB antagonist is administered orally, intravenously, enterally, or parenterally.

13. The method of claim 1, wherein administration of the Prox1 gene, Prox1 mimic, PDGF antagonist, or PDGFRB antagonist prolongs a time of survival of the patient, as compared to placebo treatment or non-PDGFRB antagonist treatment, of at least about 30 days, at least about 5 months, or at least about 7 months.

14. A method for detecting and treating a patient suffering from myxomatous mitral valve disease (MMVD), the method comprising:
identifying that the patient has at least one of: mutation or deletion of Prox1, platelet derived growth factor (PDGF) secretion is unregulated, or PDGF receptor beta (PDGFRB) signaling is unregulated; and
administering to the patient an effective amount of a pharmaceutical composition comprising at least one of: a Prox1 gene, a Prox1 mimic, a PDGF antagonist, or a PDGFRB antagonist that prevents thickening of heart valves and delays an onset of clinical symptoms of myxomatous valve disease in the patient.

15. The method of claim 14, wherein the PDGF or PDGFRB antagonist is selected from at least one of: AC710, AC710 Mesylate, AG1295, AG1296, an antagonistic human monoclonal or portion thereof targeting PDGFRB, an antagonistic human monoclonal or portion thereof targeting PDGF, avapritinib, axitinib, AZD2932, BOT-191, cediranib, celecoxib, CP 673451, crenolanib, dasatinib, Desethyl Sunitinib, DMPQ dihydrochloride, dovitinib, etoricoxib and DFU, ilorasertib, imatinib, imatinib mesylate, KG 5, lenvatinib, Linifanib, N-CP-673451, nilotinib, nintedanib, orantinib, pazopanib, PDGFRa kinase inhibitor-1, ponatibib, radotinib, regorafenib, ripretinib, sorafenib, SU 4312, SU 5402, SU14813, SU14813 maleate, SU16f, sunitinib, sunitinib malate, TAK 593, TAK-593, TG 100572, toceranib, or toceranib phosphate.

16. The method of claim 14, wherein the Prox1 gene or Prox1 mimic is an RNA, a DNA, or a derivative thereof.

17. The method of claim 14, wherein the PDGFRB antagonist is imatinib.

18. The method of claim 14, further comprising adding one or more non-active pharmaceutically acceptable ingredients selected from at least one of: buffers, excipients, binders, diluents, vehicles, lubricants, wetting, emulsifying, salts, or carriers.

19. The method of claim 14, wherein the Prox1 gene, Prox1 mimic, PDGF antagonist, or PDGFRB antagonist is administered orally as a tablet or a capsule.

20. The method of claim 14, wherein administration of the Prox1 gene, Prox1 mimic, PDGF antagonist, or PDGFRB antagonist results in one or more of the following in the patient selected from the group consisting of: effects a prolongation of a preclinical phase without exhibiting clinical symptoms of heart failure, effects a delay of onset of clinical symptoms of heart failure, increases a survival time of a treated patient as compared to placebo treatment, improves the quality of life of the treated patient, improves cardiac function/output in the treated patient, reduces sudden cardiac death of the patient due to cardiac reasons, and reduces a risk of reaching heart failure.

21. The method of claim 14, wherein the patient is a mammal selected from the group consisting of: a human, a dog, a cat, and a horse.

22. The method of claim 14, wherein the Prox1 gene, Prox1 mimic, PDGF antagonist, or PDGFRB antagonist is administered in a daily dose of 0.1, 0.5, 1, 2, 3, 4, 5, 6, 7, 8, 9 10, 12.5, 15, 20, 25, 30, 40 50, 60, 75, 80, 90 or 100 mg/kg body weight.

23. The method of claim 14, wherein a daily dose of the Prox1 gene, Prox1 mimic, PDGF antagonist, or PDGFRB antagonist is administered as two doses 0.05, 0.5, 1, 2, 3, 4, 5, 6, 7, 8, 9 10, 12.5, 15, 20, 25, 30, 40, or 50 mg/kg body weight administered every 12 hours.

24. The method of claim 14, wherein a daily dose of the PDGF inhibitor imatinib is between 100, 200, 250, 300, 400, 500, 600, 700, 750, or 800 mg per day.

25. The method of claim 14, wherein the Prox1 gene, Prox1 mimic, PDGF antagonist, or PDGFRB antagonist is administered orally, intravenously, enterally, or parenterally.

26. The method of claim 14, wherein the administration of the Prox1 gene, Prox1 mimic, PDGF antagonist, or PDGFRB antagonist further effects a prolongation of the time of survival of the patient, as compared to placebo treatment or non-PDGFRB antagonist treatment, of at least about 30 days, at least about 5 months, or at least about 7 months.

27. A method for preventing suffering from myxomatous mitral valve disease (MMVD) in a human patient, the method comprising administering to the human patient an effective amount of a pharmaceutical composition comprising a Platelet Derived Growth Factor (PDGF) antagonist or a Platelet Derived Growth Factor Receptor Beta (PDGFRB) antagonist that prevents thickening of heart valves and delays an onset of clinical symptoms of myxomatous valve disease in the human patient.
